SEQUENCE LISTING (14 sequences)

SEQ ID NO: 1 - DNA, 2010 bp, Homo sapiens
atggaggggaacaagctggaggagcaggactctagccctccacagtccactccagggctcatgaaggggaacaagcgtgaggagcaggggctgggccccgaacctgcggcgccccagcagcccacggcggaggaggaggccctgatcgagttccaccgctcctaccgagagctcttcgagttcttctgcaacaacaccaccatccacggcgccatccgcctggtgtgctcccagcacaaccgcatgaagacggccttctgggcagtgctgtggctctgcacctttggcatgatgtactggcaattcggcctgcttttcggagagtacttcagctaccccgtcagcctcaacatcaacctcaactcggacaagctcgtcttccccgcagtgaccatctgcaccctcaatccctacaggtacccggaaattaaagaggagctggaggagctggaccgcatcacagagcagacgctctttgacctgtacaaatacagctccttcaccactctcgtggccggctcccgcagccgtcgcgacctgcgggggactctgccgcaccccttgcagcgcctgagggtcccgcccccgcctcacggggcccgtcgagcccgtagcgtggcctccagcttgcgggacaacaacccccaggtggactggaaggactggaagatcggcttccagctgtgcaaccagaacaaatcggactgcttctaccagacatactcatcaggggtggatgcggtgagggagtggtaccgcttccactacatcaacatcctgtcgaggctgccagagactctgccatccctggaggaggacacgctgggcaacttcatcttcgcctgccgcttcaaccaggtctcctgcaaccaggcgaattactctcacttccaccacccgatgtatggaaactgctatactttcaatgacaagaacaactccaacctctggatgtcttccatgcctggaatcaacaacggtctgtccctgatgctgcgcgcagagcagaatgacttcattcccctgctgtccacagtgactggggcccgggtaatggtgcacgggcaggatgaacctgcctttatggatgatggtggctttaacttgcggcctggcgtggagacctccatcagcatgaggaaggaaaccctggacagacttgggggcgattatggcgactgcaccaagaatggcagtgatgttcctgttgagaacctttacccttcaaagtacacacagcaggtgtgtattcactcctgcttccaggagagcatgatcaaggagtgtggctgtgcctacatcttctatccgcggccccagaacgtggagtactgtgactacagaaagcacagttcctgggggtactgctactataagctccaggttgacttctcctcagaccacctgggctgtttcaccaagtgccggaagccatgcagcgtgaccagctaccagctctctgctggttactcacgatggccctcggtgacatcccaggaatgggtcttccagatgctatcgcgacagaacaattacaccgtcaacaacaagagaaatggagtggccaaagtcaacatcttcttcaaggagctgaactacaaaaccaattctgagtctccctctgtcacgatggtcaccctcctgtccaacctgggcagccagtggagcctgtggttcggctcctcggtgttgtctgtggtggagatggctgagctcgtctttgacctgctggtcatcatgttcctcatgctgctccgaaggttccgaagccgatactggtctccaggccgagggggcaggggtgctcaggaggtagcctccaccctggcatcctcccctccttcccacttctgcccccaccccatgtctctgtccttgtcccagccaggccctgctccctctccagccttgacagcccctccccctgcctatgccaccctgggcccccgcccatctccagggggctctgcaggggccagttcctccacctgtcctctgggggggccctga

SEQ ID NO: 2 - DNA, 1923 bp, Homo sapiens
atgcacgtgaagaagtacctgctgaagggcctgcatcggctgcagaagggccccggctacacgtacaaggagctgctggtgtggtactgcgacaacaccaacacccacggccccaagcgcatcatctgtgaggggcccaagaagaaagccatgtggttcctgctcaccctgctcttcgccgccctcgtctgctggcagtggggcatcttcatcaggacctacttgagctgggaggtcagcgtctccctctccgtaggcttcaagaccatggacttccccgccgtcaccatctgcaatgctagccccttcaagtattccaaaatcaagcatttgctgaaggacctggatgagctgatggaagctgtcctggagagaatcctggctcctgagctaagccatgccaatgccaccaggaacctgaacttctccatctggaaccacacacccctggtccttattgatgaacggaacccccaccaccccatggtccttgatctctttggagacaaccacaatggcttaacaagcagctcagcatcagaaaagatctgtaatgcccacgggtgcaaaatggccatgagactatgtagcctcaacaggacccagtgtaccttccggaacttcaccagtgctacccaggcattgacagagtggtacatcctgcaggccaccaacatctttgcacaggtgccacagcaggagctagtagagatgagctaccccggcgagcagatgatcctggcctgcctattcggagctgagccctgcaactaccggaacttcacgtccatcttctaccctcactatggcaactgttacatcttcaactggggcatgacagagaaggcacttccttcggccaaccctggaactgaattcggcctgaagttgatcctggacataggccaggaagactacgtccccttccttgcgtccacggccggggtcaggctgatgcttcacgagcagaggtcataccccttcatcagagatgagggcatctacgccatgtcggggacagagacgtccatcggggtactcgtggacaagcttcagcgcatgggggagccctacagcccgtgcaccgtgaatggttctgaggtccccgtccaaaacttctacagtgactacaacacgacctactccatccaggcctgtcttcgctcctgcttccaagaccacatgatccgtaactgcaactgtggccactacctgtacccactgccccgtggggagaaatactgcaacaaccgggacttcccagactgggcccattgctactcagatctacagatgagcgtggcgcagagagagacctgcattggcatgtgcaaggagtcctgcaatgacacccagtacaagatgaccatctccatggctgactggccttctgaggcctccgaggactggattttccacgtcttgtctcaggagcgggaccaaagcaccaatatcaccctgagcaggaagggaattgtcaagctcaacatctacttccaagaatttaactatcgcaccattgaagaatcagcagccaataacatcgtctggctgctctcgaatctgggtggccagtttggcttctggatggggggctctgtgctgtgcctcatcgagtttggggagatcatcatcgactttgtgtggatcaccatcatcaagctggtggccttggccaagagcctacggcagcggcgagcccaagccagctacgctggcccaccgcccaccgtggccgagctggtggaggcccacaccaactttggcttccagcctgacacggccccccgcagccccaacactgggccctaccccagtgagcaggccctgcccatcccaggcaccccgccccccaactatgactccctgcgtctgcagccgctggacgtcatcgagtctgacagtgagggtgatgccatctaa

SEQ ID NO: 3 - DNA, 1950 bp, Homo sapiens
atggcacccggagagaagatcaaagccaaaatcaagaagaatctgcccgtgacgggccctcaggcgccgaccattaaagagctgatgcggtggtactgcctcaacaccaacacccatggctgtcgccgcatcgtggtgtcccgcggccgtctgcgccgcctcctctggatcgggttcacactgactgccgtggccctcatcctctggcagtgcgccctcctcgtcttctccttctatactgtctcagtttccatcaaagtccacttccggaagctggattttcctgcagtcaccatctgcaacatcaacccctacaagtacagcaccgttcgccaccttctagctgacttggaacaggagaccagagaggccctgaagtccctgtatggctttccagagtcccggaagcgccgagaggcggagtcctggaactccgtctcagagggaaagcagcctagattctcccaccggattccgctgctgatctttgatcaggatgagaagggcaaggccagggacttcttcacagggaggaagcggaaagtcggcggtagcatcattcacaaggcttcaaatgtcatgcacatcgagtccaagcaagtggtgggattccaactgtgctcaaatgacacctccgactgtgccacctacaccttcagctcgggaatcaatgccattcaggagtggtataagctacactacatgaacatcatggcacaggtgcctctggagaagaaaatcaacatgagctattctgctgaggagctgctggtgacctgcttctttgatggagtgtcctgtgatgccaggaatttcacgcttttccaccacccgatgcatgggaattgctatactttcaacaacagagaaaatgagaccattctcagcacctccatggggggcagcgaatatgggctgcaagtcattttgtacataaacgaagaggaatacaacccattcctcgtgtcctccactggagctaaggtgatcatccatcggcaggatgagtatcccttcgtcgaagatgtgggaacagagattgagacagcaatggtcacctctataggaatgcacctgacagagtccttcaagctgagtgagccctacagtcagtgcacggaggacgggagtgacgtgccaatcaggaacatctacaacgctgcctactcgctccagatctgccttcattcatgcttccagacaaagatggtggagaaatgtgggtgtgcccagtacagccagcctctacctcctgcagccaactactgcaactaccagcagcaccccaactggatgtattgttactaccaactgcatcgagcctttgtccaggaagagctgggctgccagtctgtgtgcaaggaagcctgcagctttaaagagtggacactaaccacaagcctggcacaatggccatctgtggtttcggagaagtggttgctgcctgttctcacttgggaccaaggccggcaagtaaacaaaaagctcaacaagacagacttggccaaactcttgatattctacaaagacctgaaccagagatccatcatggagagcccagccaacagtattgagatgcttctgtccaacttcggtggccagctgggcctgtggatgagctgctctgttgtctgcgtcatcgagatcatcgaggtcttcttcattgacttcttctctatcattgcccgccgccagtggcagaaagccaaggagtggtgggcctggaaacaggctcccccatgtccagaagctccccgtagcccacagggccaggacaatccagccctggatatagacgatgacctacccactttcaactctgctttgcacctgcctccagccctaggaacccaagtgcccggcacaccgccccccaaatacaataccttgcgcttggagagggccttttccaaccagctcacagatacccagatgctagatgagctctga

SEQ ID NO: 4 - protein, 669 aa, Homo sapiens
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 5 - protein, 640 aa, Homo sapiens
MHVKKYLLKGLHRLQKGPGYTYKELLVWYCDNTNTHGPKRIICEGPKKKAMWFLLTLLFAALVCWQWGIFIRTYLSWEVSVSLSVGFKTMDFPAVTICNASPFKYSKIKHLLKDLDELMEAVLERILAPELSHANATRNLNFSIWNHTPLVLIDERNPHHPMVLDLFGDNHNGLTSSSASEKICNAHGCKMAMRLCSLNRTQCTFRNFTSATQALTEWYILQATNIFAQVPQQELVEMSYPGEQMILACLFGAEPCNYRNFTSIFYPHYGNCYIFNWGMTEKALPSANPGTEFGLKLILDIGQEDYVPFLASTAGVRLMLHEQRSYPFIRDEGIYAMSGTETSIGVLVDKLQRMGEPYSPCTVNGSEVPVQNFYSDYNTTYSIQACLRSCFQDHMIRNCNCGHYLYPLPRGEKYCNNRDFPDWAHCYSDLQMSVAQRETCIGMCKESCNDTQYKMTISMADWPSEASEDWIFHVLSQERDQSTNITLSRKGIVKLNIYFQEFNYRTIEESAANNIVWLLSNLGGQFGFWMGGSVLCLIEFGEIIIDFVWITIIKLVALAKSLRQRRAQASYAGPPPTVAELVEAHTNFGFQPDTAPRSPNTGPYPSEQALPIPGTPPPNYDSLRLQPLDVIESDSEGDAI

SEQ ID NO: 6 - protein, 649 aa, Homo sapiens
MAPGEKIKAKIKKNLPVTGPQAPTIKELMRWYCLNTNTHGCRRIVVSRGRLRRLLWIGFTLTAVALILWQCALLVFSFYTVSVSIKVHFRKLDFPAVTICNINPYKYSTVRHLLADLEQETREALKSLYGFPESRKRREAESWNSVSEGKQPRFSHRIPLLIFDQDEKGKARDFFTGRKRKVGGSIIHKASNVMHIESKQVVGFQLCSNDTSDCATYTFSSGINAIQEWYKLHYMNIMAQVPLEKKINMSYSAEELLVTCFFDGVSCDARNFTLFHHPMHGNCYTFNNRENETILSTSMGGSEYGLQVILYINEEEYNPFLVSSTGAKVIIHRQDEYPFVEDVGTEIETAMVTSIGMHLTESFKLSEPYSQCTEDGSDVPIRNIYNAAYSLQICLHSCFQTKMVEKCGCAQYSQPLPPAANYCNYQQHPNWMYCYYQLHRAFVQEELGCQSVCKEACSFKEWTLTTSLAQWPSVVSEKWLLPVLTWDQGRQVNKKLNKTDLAKLLIFYKDLNQRSIMESPANSIEMLLSNFGGQLGLWMSCSVVCVIEIIEVFFIDFFSIIARRQWQKAKEWWAWKQAPPCPEAPRSPQGQDNPALDIDDDLPTFNSALHLPPALGTQVPGTPPPKYNTLRLERAFSNQLTDTQMLDEL

SEQ ID NO: 7 - DNA, 1916 bp, Homo sapiens
atggctgagcaccgaagcatggacgggagaatggaagcagccacacgggggggctctcacctccaggctgcagcccagacgccccccaggccggggccaccatcagcaccaccaccaccacccaaggaggggcaccaggaggggctggtggagctgcccgcctcgttccgggagctgctcaccttcttctgcaccaatgccaccatccacggcgccatccgcctggtctgctcccgcgggaaccgcctcaagacgacgtcctgggggctgctgtccctgggagccctggtcgcgctctgctggcagctggggctcctctttgagcgtcactggcaccgcccggtcctcatggccgtctctgtgcactcggagcgcaagctgctcccgctggtcaccctgtgtgacgggaacccacgtcggccgagtccggtcctccgccatctggagctgctggacgagtttgccagggagaacattgactccctgtacaacgtcaacctcagcaaaggcagagccgccctctccgccactgtcccccgccacgagccccccttccacctggaccgggagatccgtctgcagaggctgagccactcgggcagccgggtcagagtggggttcagactgtgcaacagcacgggcggcgactgcttttaccgaggctacacgtcaggcgtggcggctgtccaggactggtaccacttccactatgtggatatcctggccctgctgcccgcggcatgggaggacagccacgggagccaggacggccacttcgtcctctcctgcagttacgatggcctggactgccaggcccgacagttccggaccttccaccaccccacctacggcagctgctacacggtcgatggcgtctggacagctcagcgccccggcatcacccacggagtcggcctggtcctcagggttgagcagcagcctcacctccctctgctgtccacgctggccggcatcagggtcatggttcacggccgtaaccacacgcccttcctggggcaccacagcttcagcgtccggccagggacggaggccaccatcagcatccgagaggacgaggtgcaccggctcgggagcccctacggccactgcaccgccggcggggaaggcgtggaggtggagctgctacacaacacctcctacaccaggcaggcctgcctggtgtcctgcttccagcagctgatggtggagacctgctcctgtggctactacctccaccctctgccggcgggggctgagtactgcagctctgcccggcaccctgcctggggacactgcttctaccgcctctaccaggacctggagacccaccggctcccctgtacctcccgctgccccaggccctgcagggagtctgcattcaagctctccactgggacctccaggtggccttccgccaagtcagctggatggactctggccacgctaggtgaacaggggctgccgcatcagagccacagacagaggagcagcctggccaaaatcaacatcgtctaccaggagctcaactaccgctcagtggaggaggcgcccgtgtactcggtgccgcagctgctctccgccatgggcagcctctacagcctgtggtttggggcctccgtcctctccctcctggagctcctggagctgctgctcgatgcttctgccctcaccctggtgctaggcggccgccggctccgcagggcgtggttctcctggcccagagccagccctgcctcaggggcgtccagctcaagccagaggccagtcagatgcccccgcctgcaggcggcacgtcagatgacccggagcccagcgggcctcatctcccacgggtgatgcttccaggggttctggcgggagtctcagccgaagagagctgggctgggccccagccccttgagactctggacacctga

SEQ ID NO: 8 - protein, 638 aa, Homo sapiens
MAEHRSMDGRMEAATRGGSHLQAAAQTPPRPGPPSAPPPPPKEGHQEGLVELPASFRELLTFFCTNATIHGAIRLVCSRGNRLKTTSWGLLSLGALVALCWQLGLLFERHWHRPVLMAVSVHSERKLLPLVTLCDGNPRRPSPVLRHLELLDEFARENIDSLYNVNLSKGRAALSATVPRHEPPFHLDREIRLQRLSHSGSRVRVGFRLCNSTGGDCFYRGYTSGVAAVQDWYHFHYVDILALLPAAWEDSHGSQDGHFVLSCSYDGLDCQARQFRTFHHPTYGSCYTVDGVWTAQRPGITHGVGLVLRVEQQPHLPLLSTLAGIRVMVHGRNHTPFLGHHSFSVRPGTEATISIREDEVHRLGSPYGHCTAGGEGVEVELLHNTSYTRQACLVSCFQQLMVETCSCGYYLHPLPAGAEYCSSARHPAWGHCFYRLYQDLETHRLPCTSRCPRPCRESAFKLSTGTSRWPSAKSAGWTLATLGEQGLPHQSHRQRSSLAKINIVYQELNYRSVEEAPVYSVPQLLSAMGSLYSLWFGASVLSLLELLELLLDASALTLVLGGRRLRRAWFSWPRASPASGASSIKPEASQMPPPAGGTSDDPEPSGPHLPRVMLPGVLAGVSAEESWAGPQPLETLDT

SEQ ID NO: 9 - DNA, 28 bp, Artificial Sequence
  Description of Artificial Sequence Primer
cgcggatccgcccataccaggtctcatg

SEQ ID NO: 10 - DNA, 30 bp, Artificial Sequence
  Description of Artificial Sequence Primer
ccggaattcctgcacatccttcaatcttgc

SEQ ID NO: 11 - DNA, 28 bp, Artificial Sequence
  Description of Artificial Sequence Primer
cgcggatccagcaggtgccactatgcac

SEQ ID NO: 12 - DNA, 28 bp, Artificial Sequence
  Description of Artificial Sequence Primer
ccgctcgaggtcttggctgctcagtgag

SEQ ID NO: 13 - DNA, 28 bp, Artificial Sequence
  Description of Artificial Sequence Primer
cgcggatcccctcaaagtcccatcctcg

SEQ ID NO: 14 - DNA, 30 bp, Artificial Sequence
  Description of Artificial Sequence Primer
ccggaattcgactagatctgtcttctcaac

(19) United States
(12) Patent Application Publication
(10) Pub. No.: US 2004/0072254 A1
(43) Pub. Date: Apr. 15, 2004

(54) High throughput cell-based assay for monitoring sodium channel activity and discovery of salty taste modulating compounds

(75) Inventors: Nicholas Callamaras, San Diego, CA (US); Hong Chang, San Diego, CA (US)

Correspondence Address: PILLSBURY WINTHROP, LLP, P.O. BOX 10500, MCLEAN, VA, 22102, US

(73) Assignee: Senomyx, Inc., LaJolla, CA (US)

(21) Appl. No.: 10/133,573

(22) Filed: Apr. 29, 2002

Related U.S. Application Data
(60) Provisional application No. 60/287,413, filed on May 1, 2001.

(51) Int. Cl. G01N 33/53; G01N 33/567; C07K 14/47

(52) U.S. Cl. 435/7.2; 530/402

(57) ABSTRACT

The present invention relates to a mammalian cell-based high-throughput assay for the profiling and screening of human epithelial sodium channel (hENaC) cloned from a human kidney c-DNA library and is also expressed in other tissues including human taste tissue. It is thought that ENaC is involved in mediating mammalian salty taste responses. Compounds that modulate ENaC function in a cell-based ENaC assay would be expected to affect salty taste in humans. The present invention also provides recombinant mammalian cells that express a functional hENaC. The assay described herein has major advantages over existing cellular expression systems, in that both mammalian cells are employed and the assay can be run in standard 96 or 384 well culture plates in high-throughput mode. In brief, the mammalian cell line HEK293T (a human embryonic kidney cell line expressing the SV40 large T-cell antigen) are transiently transfected with all three subunits of human ENaC ( [symbol] or [symbol] and □) either by Ca²⁺ phosphate or lipid-based systems. Transfected cells are seeded into 96 or 384-well culture plates, and functional expression is allowed to proceed for a minimum of 24 hours. The cells are then incubated with a membrane-potential fluorescent dye or a sodium fluorescent dye (from Molecular Devices) that provides a high-throughput, fast, simple and reliable fluorescence-based method for detecting changes in voltage across the cell membrane. The assay of the invention can reliably detect both facilitation or inhibition of hENaC function, providing a robust screen for compounds that could either enhance or block channel activity, and thereby modulate salty taste in humans.

### CROSS REFERENCE TO RELATED APPLICATION

This application claims priority from U.S. Provisional Application Serial No. 60/287,413, filed May 1, 2001, which is incorporated herein in its entirety.

### FIELD OF THE INVENTION

The present invention relates to amiloride-sensitive sodium channels and methods for using such channels to profile, screen for, and identify taste modulating compounds. More specifically, the invention relates to a human epithelial sodium channel (hENaC), the expression of hENaC in mammalian cells and the use of these cells in cells in high throughout cell-based assays to identify compounds that enhance or block hENaC function.

### BACKGROUND OF THE INVENTION

Complementary DNAs (cDNAs) encoding an amiloride-sensitive epithelial sodium channel (ENaC) have been isolated from kidney cells and expressed in a mammalian cell line. The channel expressed in this system has been shown to have similar properties to the distal renal sodium channel, i.e., high sodium selectivity, low conductance, and amiloride sensitivity. One form of the naturally occurring ENaC channel is comprised of three subunits of similar structure: alpha (OMIM Entry 600228), beta (OMIM Entry 600760), and gamma (OMIM Entry 600761). Each of the subunits is predicted to contain 2 transmembrane spanning domains, intracellular amino- and carboxy-termini, and a cysteine-rich extracellular domain. The three subunits share 32 to 37% identity in amino acid sequence.

Alternatively spliced forms of alpha-ENaC have also been identified, indicating heterogeneity of alpha subunits of amiloride-sensitive sodium channels that may account for the multiple species of proteins observed during purification of the channel (U.S. Pat. No. 5,693,756, which is herein incorporated by reference). Further, based on published electrophysiological data and the discovery that ENaC occurs in taste bud cells, a model of salty taste transduction mediated by ENaC has been constructed. As such, the use of ENaC in the identification of substances which stimulate or block salty taste perception has been suggested (U.S. Pat. No. 5,693,756, supra).

More particularly, cell-based functional expression systems commonly used for the physiological characterization of ENaC are Xenopus laevis oocytes and cultured mammalian cell lines. The oocyte system has advantages in that it allows the direct injection of multiple mRNAs, provides high levels of protein expression, and can accommodate the deleterious effects inherent in the over expression of ENaC. The drawbacks of this system are that electrophysiological recording in Xenopus oocytes is not amenable to screening large numbers of compounds and that the oocyte is not a mammalian system. Studies of the electrophysiological properties of rodent ENaC in mammalian cell lines (HEK293 and MDCK) stably expressing the channel have been reported in the literature. While these studies used mammalian cell lines, channel function was assayed using tedious electrophysiological techniques. Such approaches do not lend themselves to high throughput screening of compounds. Thus, there remains a need in the art for an assay which is amenable to high throughput screening.

### SUMMARY OF THE INVENTION

The present invention provides mammalian cells that express a functional human ENaC. The present invention also provides a mammalian cell-based high throughput assay for the profiling and screening of a sodium channel, more particularly an amiloride-sensitive epithelial sodium channel (ENaC). Such a method can be used to functionally characterize ENaC activity or to identify compounds that either enhance or block salty taste perception (herein referred to as salty taste modulators).

Accordingly, in a first aspect the invention provides recombinant mammalian cells that express a functional hENaC. In a preferred embodiment these mammalian cells will transiently express all three subunits of hENaC (alpha or delta, beta and gamma), or stably express one or more subunits or functional chimeras, variants or fragments thereof. Such mammalian cells encompass any mammalian cell capable of expressing a functional hENaC, including by way of example COS, CHO, MDCK, HEK293, HEK293T, NIH3T3, Swiss3T3 and BHK cells. In a still more preferred embodiment the invention provides HEK293T cells that express a functional hENaC.

In a second aspect, the invention provides cell-based assays that utilize mammalian cells that express a functional ENaC, preferably hENaC, to identify compounds, including e.g., small organic molecules, antibodies, peptides, cyclic peptides, lipids and nucleic acids that enhance or block ENaC function.

Preferably the assay will comprise a mammalian cell-based high throughput assay for the profiling and screening of putative modulators of an epithelial sodium channel (ENaC) comprising: (i) contacting a test cell expressing an ENaC and loaded with a membrane potential fluorescent dye or a sodium-sensitive fluorescent dye with at least one putative modulator compound in the presence of a buffer containing sodium; and (ii) monitoring changes in fluorescence of the membrane potential dye or sodium-sensitive dye for cells contacted with the putative modulator plus sodium compared to the change in fluorescence of the membrane potential dye or sodium-sensitive dye for cells contacted with sodium alone to determine the extent of ENaC modulation.

In another preferred aspect of the invention, a method for monitoring the activity of an epithelial sodium channel (ENaC) is provided comprising: (i) providing a test cell transfected with a functional ENaC; (ii) seeding the test cell in the well of a multi-well plate and incubating for a time sufficient to reach at least about 70% confluence; (iii) dye-loading the seeded test cell with a membrane potential fluorescent dye or sodium-sensitive fluorescent dye in the well of the multi-well plate; (iv) contacting the dye-loaded test cell with at least one putative modulating compound in the well of the multi-well plate; and (v) monitoring any changes in fluorescence using a fluorescence plate reader.

In a preferred embodiment of the invention (i) suitable cells, e.g., HEK293T cells are transformed or tranfected with DNA sequences encoding subunits necessary to produce a functional human ENaC; (ii) the cells are seeded onto a multi-well plates, e.g., 384 well plates, preferably to about 80% confluence; (iii) the seeded test cells are loaded with a membrane potential sensitive dye such as CC2-DMPVE or DiSBAC2(3); (iv) the dye-loaded cells are then contacted with at least one putative ENaC modulating compound; and (v) changes in cell fluorescence are monitored using a voltage intensity plate reader e.g., VIPRII (Aurora Biosciences).

In yet another aspect of the invention, a method for identifying a salty taste modulating compound is provided comprising: (i) providing a test cell transfected with a functional human ENaC; (ii) seeding the test cell in the well of a multi-well plate and incubating for a time sufficient to reach at least about 70% confluence more preferably to about 80%, confluence; (iii) dye-loading the seeded test cell with a membrane potential dye in the well of the multi-well plate; (iv) contacting the dye-loaded test cell with at least one putative modulating compound in the well of the multi-well plate; (v) monitoring any changes in fluorescence of the membrane potential dye due to modulator/ENaC interactions using a fluorescence plate reader; and (vi) identifying the at least one putative modulator as a salty taste modulating compound based on the monitored changes in fluorescence.

In a preferred embodiment of the invention (i) suitable cells, e.g., HEK293T cells are transformed as tranfected with DNA sequences encoding subunits necessary for a functional human ENaC; (ii) the cells are seeded on to multi-well plates, e.g., 384 well plates, preferably to about 80% confluence; (iii) the seeded test cells are loaded with a membrane potential sensitive dye such as CC2-DMPVE or DiSBAC2(3); (iv) the dye-based cells are then contacted with at least one putative ENaC modulating compound; and (v) changes in cell fluorescence are monitored using a voltage intensity plate reader e.g., VIPRII (Aurora Biosciences); (vi) and compounds that modulate salty taste are selected based on a change in fluorescence intensity.

In one embodiment of the invention, the ENaC can be composed of naturally occurring human ENaC subunits, one or more alternatively spliced human ENaC subunits, or a functional variant thereof. Alternatively, the ENaC can be composed of at least the alpha subunit of a naturally occurring human ENaC, or an alternatively spliced version thereof. In another embodiment, a delta subunit (such as Genebank accession U38254; see J Biol Chem, 270(46):27411-4 (1995)) or a variant thereof can substitute for the alpha subunit.

Preferably, these subunits are encoded by SEQ ID: NO.: 1, 2, 3 and 7 disclosed infra. These and other aspects of the invention will become apparent to one of skill in the art from the following detailed description, drawings, and claims.

### DETAILED DESCRIPTION OF THE DRAWINGS

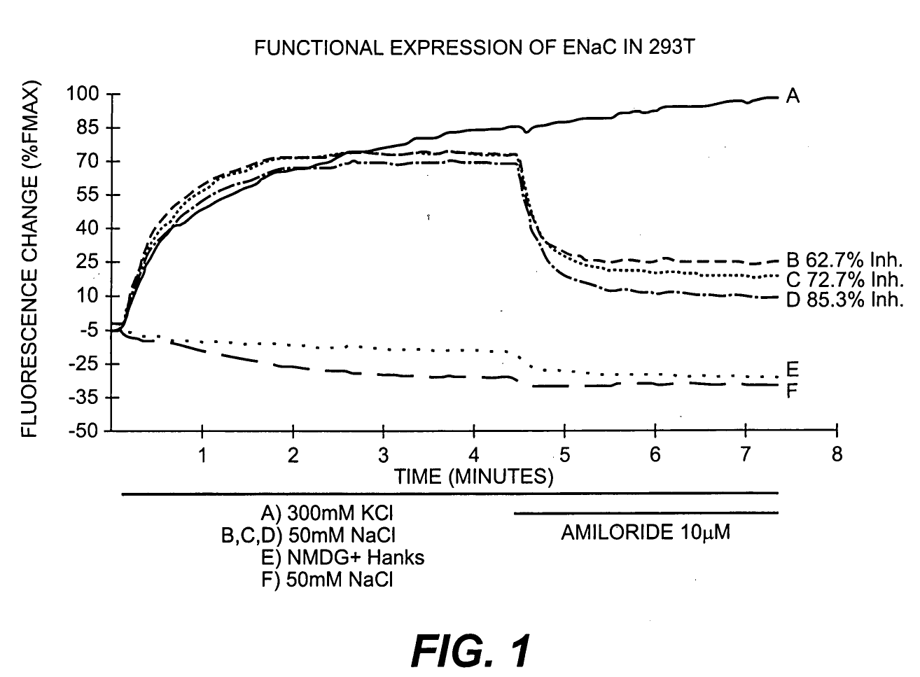

FIG. 1 illustrates the functional expression of hENaC resulting in a sodium dependent amiloride sensitive fluorescence change. Tranfection of HEK293T cells with varying 1:1:1 ratios of α, β, and γ, subunit plasmids of human kidney ENaC results in a NA⁺ dependent amiloride sensitive voltage change, as compared to mock transfected cells. A, B, C, and D were transfected with 111:1 rations of α, β, and γ plasmid at absolute levels of 4.4.1. and 0.25 respectively. E and F were mock transfected with Beta-gal and pUC. Transfection efficiency was approximately 40% and cell density was approximately 70%. All traces are from a single plate with A (n=4), B, C, D, E (n=12), and F (n=8).

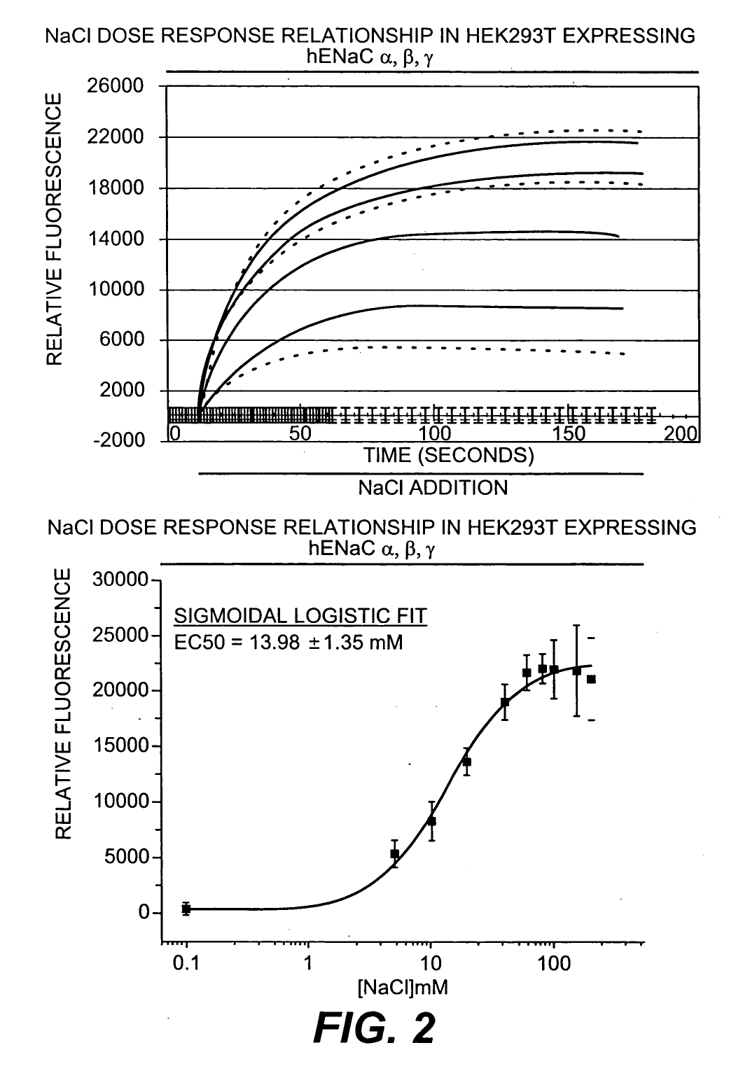

FIG. 2 illustrates the NaCl dose response relationship of HEK293T cells expressing hENaC α, β, and γ.

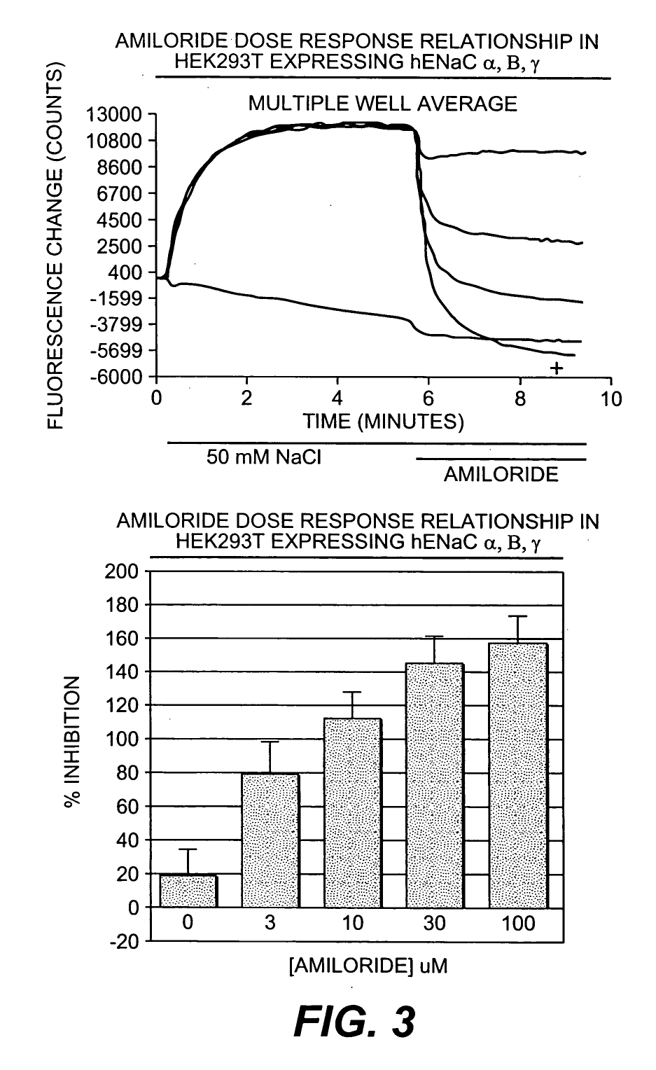

FIG. 3 illustrates the amiloride dose response relationship of HEK293T cells expressing hENaC α, β, and γ treated with 50 mM NaCl.

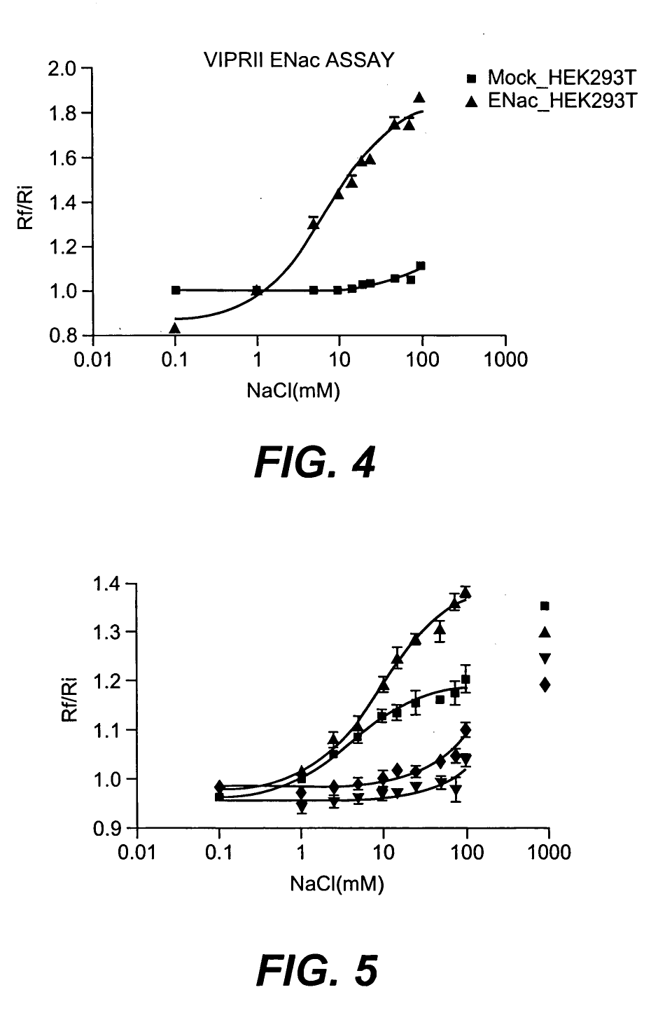

FIGS. 4 illustrates the NaCl dose response relationship of HEK293T cells expressing ENaC using a voltage imaging plate reader (VIPR). HEK293T cells were transfected with ENaC subunits expression plasmids (ENaC) or a carrier plasmid (Mock). 24 hours later cells were loaded with a membrane potential dyes and changes in cell fluorescence in response to Na+ stimulation was monitored on VIPRII (Aurora Biosciences). Only cells expressing ENaC exhibited a change in response to increases in Na⁺ concentration.

FIG. 5 also illustrates the NaCl dose response relationship of HEK2933T cells expressing human ENaC. HEK293T cells were transfected with ENaC subunits expression plasmids (ENaC) 24 hours later cells were loaded with a membrane potential dyes and changes in cell fluorescence in response to Na⁺ stimulation was monitored on VIPRII (Aurora Biosciences). Phenamil, an ENaC antagonist, inhibited Na⁺-induced changes in fluorescence. Conversely, the Compound “X”, an ENaC enhancer, increased the Na⁺-induced changes in fluorescence and this effect is inhibited by Phenamil.

### DETAILED DESCRIPTION OF THE INVENTION

The present invention provides for the first time recombinant mammalian cells that express a functional hENaC as well as a mammalian cell-based high throughout assay for the profiling and screening of an epithelial sodium channel (ENaC). More specifically, the invention provides human cell lines, in HEK293T cells that express the α, β, and γ subunits of hENaC. Also the invention provides mammalian cells that express a functional ENaC comprised of delta, beta and gamma subunits. These recombinant cells can be used to functionally characterize ENaC activity, or to identify compounds that either enhance or block salty taste perception (herein referred to as taste modulators). These compounds can be used as ingredients in foods, medicinals and beverages to enhance, modulate, inhibit or block salty taste.

However, prior to discussing the invention in more detail the following definitions are provided. It should be otherwise understood that the technical terms and phrases have their ordinary meaning, as they would be construed by use of ordinary skill in the art.

Definitions

The term “salty taste” or “salty taste perception” as used herein refers to a subject's perception or response to salt taste stimuli. As discussed above, it is believed that hENaC is involved in salty taste perception in human subjects. Such stimuli include compounds such as NaCl that elicits its active ENaCs, preferably hENaC.

The terms “ENaC” subunit protein or a fragment thereof, or a nucleic acid encoding one of three subunits of “ENaC” protein or a fragment thereof refer to nucleic acids and polypeptides, polymorphic variants, alleles, mutants, and interspecies homologues that: (1) have an amino acid sequence that has greater than about 80% amino acid sequence identity, 85%, 90%, preferably 91%, 92%, 93%, 94%, 95%, 96%, 97%, 98% or 99% or greater amino acid sequence identity, preferably over a region of over a region of at least about 25, 50, 100, 200, or 500, or more amino acids, to an amino acid sequence encoded by the nucleic acid sequence contained in SEQ ID NO:1; 2 or 3; or (2) specifically bind to antibodies, e.g., polyclonal antibodies, raised against an immunogen comprising an amino acid sequence encoded by SEQ ID NO:1, 2, or 7 or immunogenic fragments thereof, and conservatively modified variants thereof; or (3) specifically hybridize under stringent hybridization conditions to an anti-sense strand corresponding to a nucleic acid sequence encoding an ENaC protein, e.g., SEQ ID NO:1, 2, 3 or 7 or their complements, and conservatively modified variants thereof; or (4) have a nucleic acid sequence that has greater than about 80% sequence identity, 85%, 90%, preferably 91%, 92%, 93%, 94%, 95%, 96%, 97%, 98% or 99%, or higher nucleotide sequence identity, preferably over a region of at least about 25, 50, 100, 200, 500, 1000, or more nucleotides, to SEQ ID NO:1, 2, 3 or 7 or their complements, or (5) is functionally equivalent to the hENaC described herein in a sodium conductance assay when expressed in a HEK cell and tested by using two electrode whole cell electrophysiology or by the change in fluorescence of a membrane potential dye in response to sodium or lithium.

Functionally equivalent ENaC proteins include ENaC subunits with primary sequences different than those identified infra, but which possess an equivalent function as determined by functional assays, e.g., sodium conductance assays as described infra. By “determining the functional effect” refers to assaying the effect of a compound that increases or decreases a parameter that is indirectly or directly under the influence of an ENaC polypeptide e.g., functional, physical and chemical effects. Such functional effects include, but are not limited to, changes in ion flux, membrane potential, current amplitude, and voltage gating, a as well as other biological effects such as changes in gene expression of any marker genes, and the like. The ion flux can include any ion that passes through the channel, e.g., sodium or lithium and analogs thereof such as radioisotopes. Such functional effects can be measured by any means known to those skilled in the art, e.g., by the use of two electrode electrophysiology or voltage-sensitive dyes, or by measuring changes in parameters such as spectroscopic characteristics (e.g., fluorescence, absorbance, refractive index), hydrodynamic (e.g., shape), chromatographic, or solubility properties. Preferably ENaC function will be evaluated by using two electrode whole cell electrophysiology or by monitoring the change in fluorescence of a membrane potential dye in response to sodium or lithium.

“Inhibitors”, “activators”, and “modulators” of ENaC polynucleotide and polypeptide sequences are used to refer to activating, inhibitory, or modulating molecules identified using cell-based assays of ENaC polynucleotide and polypeptide sequences. Inhibitors are compounds that, e.g., bind to, partially or totally block activity, decrease, prevent, delay activation, inactivate, desensitize, or down regulate the activity or expression of ENaC proteins, e.g., antagonists. “Activators” are compounds that increase, open, activate, facilitate, enhance activation, sensitize, agonize, or up regulate ENaC protein activity. Inhibitors, activators, or modulators also include genetically modified versions of ENaC proteins, e.g., versions with altered activity, as well as naturally occurring and synthetic ligands, antagonists, agonists, peptides, cyclic peptides, nucleic acids, antibodies, antisense molecules, ribozymes, small organic molecules and the like. Such assays for inhibitors and activators include, e.g., expressing ENaC protein in cells, cell extracts, or cell membranes, applying putative modulator compounds, and then determining the functional effects on activity, as described above.

Samples or assays comprising ENaC proteins that are treated with a potential activator, inhibitor, or modulator are compared to control samples without the inhibitor, activator, or modulator to examine the extent of activation, inhibition or modulation. In one embodiment of the assay, compounds are tested for their effect on the response of cells provided with a suboptimal sodium concentration. Control cells, treated with the suboptimal concentration of sodium but lacking a compound, typically exhibit a 10-20% of the maximal response. Compounds that increase the response of the suboptimal sodium concentration above the 10-20% level are putative ENaC enhancers. In contrast, compounds that reduce the response to below 10% are putative ENaC enhancers.

The term “test compound” or “test candidate” or “modulator” or grammatical equivalents thereof as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid (e.g., a sphingolipid), fatty acid, polynucleotide, oligonucleotide, etc., to be tested for the capacity to modulate ENaC activity. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., enhancing activity, creating variants of the lead compound, and evaluating the property and activity of those variant compounds. Preferably, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“Biological sample” includes sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 80% identity, preferably 85%, 90%, 91%, 92%, 93%, 94%, 95%, 96%, 97%, 98%, 99%, or higher identity over a specified region (e.g., nucleotide sequences SEQ ID NO: 1, 2, 3 or 7), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site (www.ncbi.nlm.nih.gov) or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but those functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologous, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). As noted previously, the invention embraces cells that express ENaC subunit polypeptides having primary sequences different than those disclosed in the subject application that are functionally equivalent in appropriate assays, e.g., using whole cell sodium conductance assays described in detail infra.

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered three-dimensional structures within a polypeptide. These structures are commonly known as domains, e.g., transmembrane domains pore domains, and cytoplasmic tail domains. Domains are portions of a polypeptide that form a compact unit of the polypeptide and are typically 15 to 350 amino acids long. Exemplary domains include extracellular domains, transmembrane domains, and cytoplasmic domains. Typical domains are made up of sections of lesser organization such as stretches of □-sheet and □-helices. “Tertiary structure” refers to the complete three-dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

ENaC nucleic acid sequences also include single nucleotide polymorphisms which encode ENaC subunits that are functionally equivalent to the ENaC polypeptides disclosed herein when assayed using appropriate assays, in the sodium conductance assays described herein.

Membrane potential dyes or voltage-sensitive dyes refer to a molecule or combinations of molecules that change fluorescent properties upon membrane depolarization. These dyes can be used to detect the changes in activity of an ion channel such as ENaC expressed in a cell.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. In the present invention this typically refers to cells that have been transfected with nucleic acid sequences that encode one or more ENaC subunits.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein). The term “heterologous” when used with reference to cellular expression of a gene, cDNA, mRNA or protein indicates that the gene, cDNA, mRNA, or protein is not normally expressed in the cell or is from another species than the original source of the cells.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

Particularly, such an antibody includes one which specifically binds to an ENaC disclosed herein, or a mixture of antibodies that specifically bind such ENaC polypeptides.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to ENaC subunit proteins, e.g., the ENaC alpha, beta, gamma or delta subunits as encoded by SEQ ID NO:1, 2, 3, or 7, polymorphic variants, alleles, orthologs, and conservatively modified variants, or splice variants, or portions thereof, can be selected to obtain only those polyclonal antibodies that are specifically immunoreactive with ENaC subunit proteins i.e., ENaC alpha, beta, gamma or delta subunits, e.g., those having the amino acid sequences contained in SEQ ID NO.: 4, 5, 6 or 8, and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

Assays for Proteins that Modulate ENaC Activity

High throughput functional genomics assays can be used to identify modulators of ENaC which block, inhibit, modulate or enhance salty taste. Such assays can, e.g., monitor changes in cell surface marker expression, changes in intracellular ions, or changes in membrane currents using either cell lines or primary cells. Typically, the cells are contacted with a cDNA or a random peptide library (encoded by nucleic acids). The cDNA library can comprise sense, antisense, full length, and truncated cDNAs. The peptide library is encoded by nucleic acids. The effect of the cDNA or peptide library on the phenotype of the cells is then monitored, using an assay as described above. The effect of the cDNA or peptide can be validated and distinguished from somatic mutations, using, e.g., regulatable expression of the nucleic acid such as expression from a tetracycline promoter. cDNAs and nucleic acids encoding peptides can be rescued using techniques known to those of skill in the art, e.g., using a sequence tag.

Proteins interacting with the peptide or with the protein encoded by the cDNA (e.g., SEQ ID NO: 1, 2, or 7) can be isolated using a yeast two-hybrid system, mammalian two hybrid system, or phage display screen, etc. Targets so identified can be further used as bait in these assays to identify additional components that may interact with the ENaC channel which members are also targets for drug development (see, e.g., Fields et al., Nature 340:245 (1989); Vasavada et al., Proc. Nat'l Acad. Sci. USA 88:10686 (1991); Fearon et al., Proc. Nat'l Acad. Sci. USA 89:7958 (1992); Dang et al., Mol. Cell. Biol. 11:954 (1991); Chien et al., Proc. Nat'l Acad. Sci. USA 9578 (1991); and U.S. Pat. Nos. 5,283,173, 5,667,973, 5,468,614, 5,525,490, and 5,637,463).

Suitable cell lines that express ENaC proteins include kidney epithelial cells, lung epithelial cells, taste epithelial cells and other mammalian epithelial cells, preferably human.

Isolation of Nucleic Acids Encoding ENaC Proteins

This invention relies on routine techniques in the field of recombinant genetics. Basic texts disclosing the general methods of use in this invention include Sambrook and Russell, Molecular Cloning, A Laboratory Manual (3^{rd} ed. 2001); Kriegler, Gene Transfer and Expression: A Laboratory Manual (1990); and Current Protocols in Molecular Biology (Ausubel et al., eds., 1994)).

Nucleic acids that encode ENaC subunits, polymorphic variants, orthologs, and alleles that are substantially identical to an amino acid sequence encoded by SEQ ID NO: 1, 2, 3 or 7 as well as other ENaC family members, can be isolated using ENaC nucleic acid probes and oligonucleotides under stringent hybridization conditions, by screening libraries. Alternatively, expression libraries can be used to clone ENaC subunit protein, polymorphic variants, orthologs, and alleles by detecting expressed homologous immunologically with antisera or purified antibodies made against human ENaC or portions thereof.

To make a cDNA library, one should choose a source that is rich in ENaC RNA. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, Gene 25:263-269 (1983); Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science 196:180-182 (1977). Colony hybridization is carried out as generally described in Grunstein et al., Proc. Natl. Acad. Sci. USA., 72:3961-3965 (1975).

An alternative method of isolating ENaC subunit nucleic acid and its orthologs, alleles, mutants, polymorphic variants, and conservatively modified variants combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of human ENaC directly from mRNA, from cDNA, from genomic libraries or cDNA libraries. Degenerate oligonucleotides can be designed to amplify ENaC homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of ENaC encoding mRNA in physiological samples, for nucleic acid sequencing, or for other purposes. Genes amplified by the PCR reaction can be purified from agarose gels and cloned into an appropriate vector.

Gene expression of ENaC subunits can also be analyzed by techniques known in the art, e.g., reverse transcription and amplification of mRNA, isolation of total RNA or poly A⁺ RNA, northern blotting, dot blotting, in situ hybridization, RNase protection, high density polynucleotide array technology, e.g., and the like.

Nucleic acids encoding ENaC subunit proteins can be used with high-density oligonucleotide array technology (e.g., GeneChip™) to identify ENaC protein, orthologs, alleles, conservatively modified variants, and polymorphic variants in this invention. In the case where the homologs being identified are linked to modulation of T cell activation and migration, they can be used with GeneChip™ as a diagnostic tool in detecting the disease in a biological sample, see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998).

The genes encoding ENaC subunits preferably human ENaC subunits are typically cloned into intermediate vectors before transformation into prokaryotic or eukaryotic cells for replication and/or expression. These intermediate vectors are typically prokaryotic vectors, e.g., plasmids, or shuttle vectors.

Expression in Prokaryotes and Eukaryotes

To obtain high level expression of a cloned gene, such as those cDNAs encoding hENaC subunit, one typically subclones the hENaC subunit nucleic acid sequence into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al., and Ausubel et al, supra. Bacterial expression systems for expressing the ENaC subunit protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. In a preferred embodiment retroviral expression systems are used in the invention. In another embodiment transient expression systems are utilized using plasmid-based vectors that are commercially available such as pcDNA 3 and derivatives thereof

Selection of the promoter used to direct expression of a heterologous nucleic acid depend on the particular application. The promoter is preferably positioned about the same distance from the heterologous transcription start site, as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the ENaC subunit encoding nucleic acid in host cells. A typical expression cassette thus contains at least one promoter operably linked to a nucleic acid sequence encoding a ENaC subunit(s) and signals required for efficient polyadenylation of the transcript, ribosome binding sites, and translation termination. Additional elements of the cassette may include enhancers and, if genomic DNA is used as the structural gene, introns with functional splice donor and acceptor site.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags may be included in an expression cassette for nucleic acid rescue. Markers such as fluorescent proteins, green or red fluorescent protein, □-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal.

In one embodiment, the vectors of the invention have a regulatable promoter, e.g., tet-regulated systems and the RU-486 system (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a ENaC encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of ENaC protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, lipids optimized for DNA transfection, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one ENaC subunit gene into a host cell, preferably mammalian capable of expressing functional ENaC.

After the expression vector is introduced into the cells, the transfected cells are cultured under conditions favoring expression of ENaC subunit(s). In one embodiment, the cells are transiently transfected with all three hENaC genes using lipid-based transfection and cultured for 24-48 hours prior to performing the screen for ENaC modulators.

Assays for Modulators of ENaC Protein

A. Assays

Modulation of an ENaC protein can be assessed using a variety of assays; preferably cell-based models as described above. Such assays can be used to test for inhibitors and activators of ENaC, which modulate, block, enhance or inhibit salty taste perception.

Preferably, the ENaC will be comprised of three subunits, alpha (or delta), beta and gamma and preferably the human ENaC subunit encoded by the encoded by SEQ ID NO: 1, 2, 3 or 7 or a human ortholog a conservatively modified variant thereof. Alternatively, the ENaC of the assay will be derived from a non-human epithelial cell. Generally, the amino acid sequence identity of each respective subunit will be at least 80%, preferably at least 85%, or 90%, most preferably at least 95%, e.g., 96%, 97%, 98% or 99% to the polypeptide encoded by SEQ ID NO: 1, 2, 3 or 7.

Measurement of the effect of a candidate comprised or an ENaC protein or cell expressing ENaC protein, either recombinant or naturally occurring, can be performed using a variety of assays, as described herein. Preferably to identify molecules capable of modulating ENaC, assays are performed to detect the effect of various candidate modulators on ENaC activity in a mammalian cell that expresses a functional ENaC.

The channel activity of ENaC proteins can be assayed using a variety of assays to measure changes in ion fluxes including patch clamp techniques, measurement of whole cell currents, radiolabeled ion flux assays, and fluorescence assays using voltage-sensitive dyes (see, e.g., Vestergarrd-Bogind et al., J. Membrane Biol. 88:67-75 (1988); Daniel et al., J. Pharmacol. Meth. 25:185-193 (1991); Hoevinsky et al., J. Membrane Biol. 137:59-70 (1994)) and ion-sensitive dyes. For example, nucleic acids encoding one or more subunits of an ENaC protein or homologue thereof can be injected into Xenopus oocytes. Channel activity can then be assessed by measuring changes in membrane polarization, i.e., changes in membrane potential. One means to obtain electrophysiological measurements is by measuring currents using patch clamp techniques, e.g., the “cell-attached” mode, the “inside-out” mode, and the “whole cell” mode (see, e.g., Ackerman et al., New Engl. J. Med. 336:1575-1595, 1997). Whole cell currents can be determined using standard methodology such as that described by Hamil et al., PFlugers. Archiv. 391:185 (1981).

Channel activity is also conveniently assessed by measuring changes in intracellular ion levels for example using ion sensitive dyes.

The activity of ENaC polypeptides can be also assessed using a variety of other assays to determine functional, chemical, and physical effects, e.g., measuring the binding of ENaC polypetides to other molecules, including peptides, small organic molecules, and lipids; measuring ENaC protein and/or RNA levels, or measuring other aspects of ENaC polypeptides, e.g., transcription levels, or physiological changes that affects ENaC activity. When the functional consequences are determined using intact cells or animals, one can also measure a variety of effects such as changes in cell growth or pH changes or changes in intracellular second messengers such as IP3, cGMP, or cAMP, or components or regulators of the phospholipase C signaling pathway. Such assays can be used to test for both activators and inhibitors. Modulators thus identified are useful for, e.g., as flavorants in foods, beverages and medicines.

Cell-Based Assays

In another embodiment, at least one ENaC subunit protein is expressed in a cell, and functional, e.g., physical and chemical or phenotypic, changes are assayed to identify ENaC modulators. Cells expressing ENaC proteins can also be used in binding assays. Any suitable functional effect can be measured, as described herein. For example, changes in membrane potential, changes in intracellular ion levels, and ligand binding are all suitable assays to identify potential modulators using a cell based system. Suitable cells for such cell-based assays include both primary cells, e.g., taste epithelial cells that expresses an ENaC protein and cultured cell lines such as HEK293T cells that express an ENaC. The ENaC protein can be naturally occurring or recombinant. Also, as described above, fragments of ENaC proteins or chimeras with ion channel activity can be used in cell based assays.

In another embodiment, cellular ENaC polypeptide levels are determined by measuring the level of protein or mRNA. The level of ENaC protein or proteins related to ENaC ion channel activation are measured using immunoassays such as western blotting, ELISA and the like with an antibody that selectively binds to the ENaC polypeptide or a fragment thereof. For measurement of mRNA, amplification, e.g., using PCR, LCR, or hybridization assays, e.g., northern hybridization, RNase protection, dot blotting, is preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, ENaC expression can be measured using a reporter gene system. Such a system can be devised using an ENaC protein promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

In another embodiment, a functional effect related to signal transduction can be measured. An activated or inhibited ENaC will alter the properties of target enzymes, second messengers, channels, and other effector proteins. Assays for ENaC activity include cells that are loaded with ion or voltage sensitive dyes to report channel activity, e.g., by observing membrane depolarization or sodium influx. Assays for determining activity of such receptors can also use known antagonists for ENaC, such as amiloride or phenamil, as controls to assess activity of tested compounds. In assays for identifying modulatory compounds (e.g., agonists, antagonists), changes in the level of ions in the cytoplasm or membrane potential will be monitored using an ion sensitive or membrane potential fluorescent indicator, respectively. Among the ion-sensitive indicators and voltage probes that may be employed are those disclosed in the Molecular Probes 2002 Catalog: (www.probes.com). and specific compounds disclosed infra.

Animal Models

Animal models that express hENaC also find use in screening for modulators of salty taste. Similarly, transgenic animal technology including gene knockout technology, for example as a result of homologous recombination with an appropriate gene targeting vector, or gene overexpression, will result in the absence or increased expression of the ENaC protein. The same technology can also be applied to make knockout cells. When desired, tissue-specific expression or knockout of the ENaC protein may be necessary. Transgenic animals generated by such methods find use as animal models of responses to salty taste stimuli.

Knockout cells and transgenic mice can be made by insertion of a marker gene or other heterologous gene into an endogenous ENaC gene site in the mouse genome via homologous recombination. Such mice can also be made by substituting an endogenous ENaC with a mutated version of the ENaC gene, or by mutating an endogenous.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

B. Modulators

The compounds tested as modulators of ENaC protein can be any small organic molecule, or a biological entity, such as a protein, e.g., an antibody or peptide, a sugar, a nucleic acid, e.g., an antisense oligonucleotide or a ribozyme, or a lipid. Alternatively, modulators can be genetically altered versions of an ENaC protein. Typically, test compounds will be small organic molecules, peptides, lipids, and lipid analogs. Preferably, the tested compounds are safe for human consumption.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including ChemDiv (San Diego, Calif.), Sigma-Aldrich (St. Louis, Mo.), Fluka Chemika-Biochemica-Analytika (Buchs Switzerland) and the like.

In the preferred embodiment, high throughput screening methods involve providing a small organic molecule or peptide library containing a large number of potential ENaC modulators (potential activator or inhibitor compounds). Such “chemical libraries” are then screened in one or more assays, as described herein, to identify those library members (particular chemical species or subclasses) that display a desired characteristic activity. The compounds thus identified can serve as conventional “lead compounds” or can themselves be used as potential or actual products.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No, 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Foods and Beverage Compositions Containing Compound Identified Using Disclosed Assays

The compounds identified using disclosed assays, in particular the fluorescence cell-based assay disclosed in the example, are potentially useful as ingredients or flavorants in ingestible compositions, i.e., foods and beverages as wells as orally administered medicinals. Compounds that modulate or enhance salty taste perception can be used alone or in combination as flavorants in foods or beverages. In the preferred application, the modulator will be incorporated into a food or beverage with a reduced level of sodium and the salty taste of the resulting product will be similar to that of the high sodium product. Examples of such foods and beverages include snack foods such as pretzels, potato chips, crackers, soups, dips, soft drinks, packaged meat products, among others.

Alternatively, compounds that block or inhibit salty taste perception can be used as ingredients or flavorants in foods that naturally contain high salt concentrates in order to block or camouflage the salty taste thereof.

The amount of such compound(s) will be an amount that yields the desired degree of salty taste perception. Of course compounds used in such applications will be determined to be safe for human consumption.

Preferred Embodiment

As disclosed supra preferably, the invention will comprise contacting a test cell expressing a functional ENaC with at least one putative modulator compound in the presence of a membrane potential dye, and monitoring the activity of the ENaC expressed by the test cell to determine the extent of ENaC modulation. The method can further comprise evaluating the putative modulator compound for in vivo effects on salty taste perception (e.g., performing tasting experiments to determine the in vivo effect on salty taste perception). In one embodiment, cDNAs encoding the ENaC subunits are cloned from human kidney cell cDNA, human lung cell cDNA, or human taste cell cDNA. As mentioned above, native ENaC is a multimeric protein consisting of three subunits (alpha or delta, beta, and gamma). ENaC functions as a constitutively active Na⁺ selective cation channel, is found in taste buds as well as other tissues, and is a candidate human salt receptor underlying the physiological perception of salt taste.

In a preferred embodiment, such a method is carried out in a high throughput assay format using multi-well plates and a fluorescence intensity plate reader (e.g., Aurora Biosciences VIPR instrument or Molecular Device's FLIPR instrument). The test cells may be seeded, dye-loaded, contacted with the test compounds, and monitored in the same multi-well plate. Such an assay format can reliably detect both activation or inhibition of ENaC function, providing a robust screen for compounds that could either enhance or block channel activity. The assay described above has been optimized to identify ENaC enhancers. The assay described herein thus has advantages over existing assays, such as those described above, in that a human ENaC is utilized, mammalian cells are employed and the assay can be run in standard multi-well (e.g., 96, 384, or 1536 well) plates in high-throughput mode.

In one aspect of the invention, mammalian cells will be produced that functionally express at least the alpha (or delta) subunit of ENaC. In preferred embodiments, all three subunits of hENaC (α or δ, β, and γ) are expressed either transiently or stably. The ENaC subunit(s) employed can be naturally occurring forms, variants containing SNPs, alternatively spliced forms, combinations of forms or any functional variants known in the art (see e.g., accession numbers P37088, P51168, P51170, and P51172). Preferably, the ENaC will be comprised of the human alpha, beta and gamma ENaC subunits encoded by the nucleic acid sequence in SEQ ID NO. 1, 2, 3 or the human beta, gamma and delta ENaC subunits encoded by SEQ ID NO. 2, 3 and 7. The mammalian cells can be any type known in the art such as COS, CHO, BHK, MDCK, HEK293, or HEK293T (human embryonic kidney cells expressing the large T-cell antigen). Preferably, the cell is HEK293T. The cells can be transfected using standard methods known in the art, such as but not limited to Ca²⁺ phosphate or lipid-based systems, or methods previously mentioned.

In a preferred embodiment of the invention, transfected cells are seeded into multi-well culture plates. Functional expression is then allowed to proceed for a time sufficient to reach at least about 70% confluence, more preferably to at least about 80% confluence or to form a cell layer dense enough to withstand possible fluid perturbations caused by compound addition. Generally, an incubation time of at least 24 hours will be sufficient, but can be longer as well. The cells are then washed to remove growth media and incubated with a membrane-potential dye for a time sufficient to allow the dye to equilibrate across the plasma membranes of the seeded cells. One of skill in the art will recognize that the dye loading conditions are dependent on factors such as cell type, dye type, incubation parameters, etc. In one embodiment, the dye may be used at about 2 μM to about 5 μM of the final concentration. Further, the optimal dye loading time may range from about 30 to about 60 minutes at 37° C. for most cells. In the preferred embodiment, the membrane potential dyes are from Molecular Devices (cat# R8034). In other embodiments, suitable dyes could include single wavelength-based dyes such as DiBAC, DiSBAC (Molecular Devices), and Di-4-ANEPPS (Biotium), or dual wavelength FRET-based dyes such as DiSBAC2, DiSBAC3, and CC-2-DMPE (Aurora Biosciences). [Chemical Names—Di-4-ANEPPS (Pyridinium, 4-(2-(6-(dibutylamino)-2-naphthalenyl)ethenyl)-1-(3-sulfopropyl)-, hydroxide, inner salt), DiSBAC4(2) (bis-(1,2-dibarbituric acid)-trimethine oxanol), DiSBAC4(3) (bis-(1,3-dibarbituric acid)-trimethine oxanol), CC-2-DMPE (Pacific Blue™ 1,2-ditetradecanoyl-sn-glycero-3-phosphoethanolamine, triethylammonium salt) and SBFI-AM (1,3-Benzenedicarboxylic acid, 4,4′-[1,4,10-trioxa-7,13-diazacyclopentadecane-7,13-diylbis(5-methoxy-6,12-benzofurandiyl)]bis-, tetrakis[(acetyloxy)methyl] ester;].

In one embodiment, the dye-loaded cells are then contacted with test compounds (or controls), and the cell cultures are monitored using standard fluorescence analysis instrumentation such as or VIPR or FLIPR®. The addition of NaCl or other test compounds which pharmacologically act on ENaC elicit a change in membrane potential which is then detected as a change in the resting fluorescence in a standard fluorescence intensity plate reader (e.g., FLIPR) or voltage intensity plate reader (e.g. VIPR). As such, the method of the present invention can be used to identify taste modulating compounds by monitoring the activity of ENaC in the test cells through fluorescence. For instance, a decrease in fluorescence may indicate a taste (salty) blocker, while an increase in fluorescence may indicate a taste (salty) enhancer.

Having generally described the invention, the same will be more readily understood by reference to the following examples, which are provided by way of illustration and are not intended as limiting. It is understood that various modifications and changes can be made to the herein disclosed exemplary embodiments without departing from the spirit and scope of the invention.

### EXAMPLE 1

DNA sequences encoding the alpha, beta and gamma subunit of a human ENaC expressed in human taste cells were cloned from human kidney cells by RT-PCR.

Methods for Cloning Human Epithelium Sodium Channel Subunit DNA Sequences (ENaCs)

Human ENaC cDNAs for □, □ and □ ENaC were amplified from human kidney cDNA (Origene Technologies Inc.) by PCR using the following primer pairs, respectively: 5′ CGC GGA TCC GCC CAT ACC AGG TCT CAT G 3′ and 5′ CCG GAA TTC CTG CAC ATC CTT CAA TCT TGC 3′; 5′ CGC GGA TCC AGC AGG TGC CAC TAT GCA C 3′ and CCG CTC GAG GTC TTG GCT GCT CAG TGA G 3′; 5′ CGC GGA TCC CCT CAA AGT CCC ATC CTC G 3′ and 5′ CCG GAA TTC GAC TAG ATC TGT CTT CTC AAC 3′. The primers were designed to be complementary to 5′ and 3′-untranslated region sequence in order to retain the endogenous translation initiation signal, and they introduced terminal restriction endonuclease sites that were used to clone amplified ENaC cDNAs into the mammalian expression vector pcDNA3 (Invitrogen) for functional expression experiments. The cloned ENaC cDNAs were sequenced and compared to ENaC sequences in public DNA databanks. Each cloned subunit is a composite of polymorphisms present in different databank alleles; that is, every polymorphism in each cloned subunit identified by pairwise comparison of the cloned subunit to a databank allele could be found in another databank allele. In addition, polymorphisms in cloned □ ENaC were verified by sequencing of cloned cDNAs amplified in independent PCR experiments.

The nucleic acid sequences encoding cloned sequences alpha, beta and gamma hENaC subunits are respectively contained in SEQ ID NO: 1, SEQ ID NO: 2 and SEQ ID NO: 3 and the corresponding amino acid sequences in SEQ ID NO: 4, 5 and 6. Each of these DNA sequences was inserted into the expression vector pcDNA3 to produce alpha, beta and gamma subunit plasmids that express human ENaC subunit polypeptides. Also, the nucleic acid sequence for the human amiloride sensitive sodium channel delta subunit (□NaCh) is contained in SEQ ID NO: 7, which functions equivalently to the ENaC alpha subunit. The amino acid sequence for the delta subunit is contained in SEQ ID NO: 8. HEK293T cells were transiently transfected via Ca²⁺ phosphate with 1:1:1 weight ratios of (α, β, and γ subunit plasmids expressing human ENaC. Such transfection resulted in a Na⁺ dependent amiloride sensitive fluorescence change, as compared to mock-transfected cells. With reference to FIG. 1, samples A, B, C, and D were transfected with 1:1:1: ratios of α, β, and γ subunit plasmids at absolute levels of 4, 4, 1, and 0.25 micrograms, respectively. Samples E and F were mock transfected with Beta-gal and pUC DNAs. Transfection efficiency was approximately 40% and cell density was approximated 70%. Cells were analyzed using a FLIPR I (Molecular Devices) instrument using a membrane-potential fluorescent dye. All traces shown are from a single plate with A (n=4), B, C, D, E, (n=12), and F (n=8).

As depicted in FIGS. 1, 2, and 3, sodium-dependant amiloride-sensitive changes in resting potential (hENaC responses) were not significantly affected in untransfected HEK293T cells. Further, such resting potential changes were greatly enhanced in cells transfected with all three subunits of the hENaC compared to cells tranfected with only the alpha subunit of hENaC (data not shown). Moreover, the ability of NaCl to induce membrane potential changes, and the effect of amiloride to block hENaC channel activity follow dose response-relationships similar to that reported in the literature using low throughput electrophysiological recording.

### EXAMPLE 2

DNA sequences encoding the alpha, beta and gamma subunits of a human ENaC, SEQID 1, 2, and 3, respectively, were each cloned into the expression vector pcDNA3 to produce alpha, beta and gamma subunit plasmids that express human ENaC subunit polypeptides. HEK293T cells were transiently transfected via lipofection with 1:1:1 weight ratios of α, β, and γ subunit plasmids expressing human ENaC (2□g of each subunit/20 million cells). Transfected cells were plated into 384-well plates and analyzed on a VIPRII Instrument (Aurora Biosciences) using voltage-sensitive fluorescent dyes. Cells expressing ENaC exhibited a Na⁺ dependent fluorescence change, as compared to mock-transfected cells (FIG. 1). In FIG. 2, the Na⁺-dependent fluorescence change is totally abolished by Phenamil, a known ENaC antagonist. Conversely, another compound was found to increase the Na⁺⁻dependent fluorescence change but this effect is abolished by Phenamil. This compound is therefore theorized to be an ENaC enhancer, as it furthermore acts opposite to Phenamil in this assay for ENaC function.

Methods and Materials for Example 2:

1. All materials used are identified below in the “Materials Section”.

2. HEK293T cells are grown to 80% confluence and dissociated from the culture dishes with an enzymatic solution (Trypsin/EDTA) for 3 minutes at 37° C. Detached cells are analyzed for density and viability using a bench top flow cytometer (Guava; Guava Technologies). Cells with less than 85% viability are discarded from the experiment.

[The procedures herein are conditions for transfection of HEK293T cells equivalent to ten screening 384-well plates (200,000,000 cells). These conditions can be altered e.g., by increasing or decreasing cell confluency by use of different size multi-well plates etc.]

3. Dissociated cells are washed and recovered in their culture medium (complete) at a density of ˜1,000,000 cells/ml. Mammalian expression plasmid DNAs encoding the human ENaC subunits are mixed in an eppendorf in an equal ratio (10 ug □; 10 ug □ and 10 ug □/20,000,000 cells). 170□g of carrier plasmid DNA (pUC-18) is then added to the DNA mix (for a total of 200□g DNA/200,000,000 cells). 557 ul of the transfection reagent TransIT (Panvera Corporation) is added to 20 ml of culture medium exempt of serum and antibiotic. The DNA solution is then added to the Transit solution and the DNA-lipid solution is incubated at room temperature. After 60 minutes, the DNA-lipid complexes are transferred into the cell solution and volume is adjusted to 320 ml with complete cell culture medium for a final density of 635,000 viable cells/ml. (As discussed previously, the alpha subunit DNA may be interchanged with the delta subunit DNA and used to produce recombinant cells that express a functional ENaC comprised of the beta, gamma and delta ENaC subunits.)

4. Black 384-well poly-D-lysine clear bottom screening plates (Becton Dickinson) are coated with 40□l/well of a Matrigel solution (20□g/ml; Collaborative Biomedical Products) for 1 hour at room temperature. Coating solution is removed and plates are kept at room temperature until cell plating.

5. The cell/DNA solution is plated with a Multidrop into 384 well plates at a density of 50,000 cells/well (80□l/well).

6. 27 hours after plating, cells are washed and loaded with the membrane potential sensitive dyes (CC2-DMPE and DiSBAC2(3)) as described below.

7. Cells are stimulated with 200□M compounds ([2×]) and read on line using a Voltage Intensity Plate Reader (VIPRII; Aurora Biosciences Corporation). Other concentrations of compounds can be used in the assay. Buffer preparation and plate layout are described below in the VIPR. The assay is performed at “room temperature”, typically about 22° C., but can also be performed at other temperatures by preheating or cooling the cells and reagents prior to addition of compounds.

Materials

1. HEK 293T cells growing on 150 cm² flask (Becton Dickinson 0.2 um vented Blueplug seal cap) (37° C., 6% CO₂)

2. Dulbecco's Modified Eagle Medium (DMEM) (cat#11965-092 Gibco BRL) (Kept at 4° C.)

3. DMEM with HEPES (DMEMH) (cat#12430-054, Gibco BRL) (Kept at 4° C.)

4. Foetal Bovine serum (FBS) (cat#10082-147, Gibco BRL) (Kept in −20° C.)

5. Trypsin EDTA (1×) (cat#25200-072 Gibco-BRL) (Kept in −-20° C.)

6. TransIT-293 (cat#MIR2705, Panvera) (Kept in 4° C.)

7. α, β and γ ENaC DNA preparations (1 μg/μL each) (Kept in 4° C.)

8. pUC18 carrier DNA ((1 μg/μL) (Kept in 4° C.)

9. Matrigel (cat#40230,Collaborative Biomedical Products)

Cell Loading

HBSS—Hank's Buffered Saline Solution

| | | |
|---|---|---|
| DiSBAC₂(3) | 5 mM in 100% DMSO | 2.5□M |
| ESS-CY4 or VABSC-1 | 200 mM in dH₂O | 350□M |

VIPR NMDG BUFFER—see formula in “VIPR Plate Layout” section below:

| | | To Make | | |
|---|---|---|---|---|
| | | Volume | | |
| Components | 10 ml | 50 ml | 100 ml | 200 |
| CC2-DMPE (□l) | 20 | 100 | 200 | 400 |
| Pluronic (□l) | 20 | 100 | 200 | 400 |
| HBSS (ml) | 10 | 50 | 100 | 200 |
| DiSBAC₂(3) (□l) | 5 | 25 | 50 | 100 |
| ESS (□l) | 17.5 | 87.5 | 175 | 350 |
| VIPR NMDG Buffer (ml) | 10 | 50 | 100 | 200 |

Preparation of CC2-DMPE Loading Buffer

1. Mix equal volumes of the CC2-DMPE stock solution and Pluronic F127.

2. Add the CC2-DMPE/Pluronic mix to HBSS while vortexing.

Loading of cells with CC2-DMPE

1. Remove cells from CO₂ incubator.

2, Look for variation of density/well

3. Prime EMBLA with HBSS

4. Wash cells with HBSS 3×80 ul to remove residual growth medium and serum

5. Add 40 μL of 10 μM CC2-DMPE loading buffer to each well

6, Look for variation of density/well

7. Incubate for 30 minutes at room temperature in the dark.

Preparation of DiSBAC₂(3) loading buffer (can be done during CC2 incubation)

1. Mix DiSBAC₂(3) and ESS-CY4 or VABSC-1, plus double volume of PluronicF127 of DiSBAC2(3)

2. Add the above mix to VIPR NMDG BUFFER, vortex

Loading of cells with DiSBAC2(3) loading buffer

1. Prime EMBLA with NMDG buffer

2. Wash CC2-DMPE-loaded cells using VIPR NMDG buffer as the wash buffer, 3×80□l/well

3. Add 40□l of 2.5□M DiSBAC2(3), 350□M ESS-CY4 or VABSC-1 loading buffer to each well

4, Look for variation of density/well

5. Incubate for 20 minutes at room temperature in the dark before running on VIPR II

VIPR Plate Layout

[image]

While the invention has been described by way of example embodiments, it is understood that the words which have been used herein are words of description, rather than words of limitation. Changes may be made, within the purview of the appended claims, without departing from the scope and spirit of the invention in its broader aspects. Although the invention has been described herein with reference to particular means, materials, and embodiments, it is understood that the invention is not limited to the particulars disclosed. The invention extends to all equivalent structures, means, and uses which are within the scope of the appended claims.

SEQ ID NO: 1

Length 2010 nucleotides

DNA

Human hENaC alpha clone #3-1-1 coding sequence

atggaggggaacaagctggaggagcaggactctagccctccacagtccactccagggctcatgaagggg
aacaagcgtga
ggagcaggggctgggccccgaacctgcggcgccagcagcccacggcggaggaggaggccctgatc
gagttccaccgctcctaccgagagctcttcgagttcttctgcaacaacaccaccatccacggcgccatccgcc
tggtgtgctcccagcacaac
cgcatgaagacggccttctgggcagtgctgtggctctgcacctttggcatgatgtactggcaattcggcctgcttt
tcgg
agagtacttcagctaccccgtcagcctcaacatcaacctcaactcgacaagctcgtcttccccgcagtgacc
atctgca
ccctcaatccctacaggtacccggaaattaaagaggagcttgaggagctggaccgcatcacagagcaga
cgctctttgac
ctgtacaaatacagctccttcaccactctcgtggccggctccgcagccgtcgcgacctgcgggggactctgc
cgcaccc
cttgcagcgcctgagggtcccgccccgcctcacggggcccgtcgagcccgtagcgtggcctccagcttgc
gggacaaca
acccccaggtggactggaaggactggaagatcggcttccagctgtgcaaccagaacaaatcggactgcttc
taccagaca
tactcatcaggggtggatgcggtgagggagtggtaccgcttccactacatcaacatcctgtcgaggctgccag
agactct
gccatccctggaggaggacacgctgggcaacttcatcttcgcctgccgcttcaaccaggtctcctgcaaccag
gcgaatt
actctcacttccaccacccgatgtatggaaactgctatactttcaatgacaagaacaactccaacctctggatgt
cttcc
atgcctggaatcaacaacggtctgtccctgatgctgcgcgcagagcagaatgacttcaftcccctgctgtccac
agtgac
tggggcccgggtaatggtgcacgggcaggatgaacctgcctttatggatgatggtggctttaacttgcggcctg
gcgtgg
agacctccatcagcatgaggaaggaaaccctggacagacttgggggcgattatggcgactgcaccaagaa
tggcagtgat
gttcctgttgagaacctttacccttcaaagtacacacagcaggtgtgtattcactcctgcttccaggagagcatg
atcaa
ggagtgtggctgtgcctacatcttctatccgcggccccagaacgtggagtactgtgactacagaaagcacagt
tcctggg
ggtactgctactataagctccaggttgacttctcctcagaccacctgggctgtttcaccaagtgccggaagccat
gcagc
gtgaccagctaccagctctctgctggttactcacgatggccctcggtgacatcccaggaatgggtcttccagat
gctatc
gcgacagaacaattacaccgtcaacaacaagagaaatggagtggccaaagtcaacatcttcttcaaggag
ctgaactaca
aaaccaattctgagtctccctctgtcacgatggtcaccctcctgtccaacctgggcagccagtggagcctgtgg
ttcggc
tcctcggtgttgtctgtggtggagatggctgagctcgtctttgacctgctggtcatcatgttcctcatgctgctccga
ag
gttccgaagccgatactggtctccaggccgagggggcaggggtgctcaggaggtagcctccaccctggcat
cctcccctc
cttcccacttctgcccccaccccatgtctctgtccttgtcccagccaggccctgctccctctccagccttgacagc
ccct
ccccctgcctatgccaccctgggcccccgcccatctccagggggctctgcaggggccagttcctccacctgtc
ctctggg
ggggccctga

SEQ ID NO: 2

Length 1923 nucleotides

DNA

Human hENaC beta clone #5 coding sequence

atgcacgtgaagaagtacctGctgaagggcctgcatcggctgcagaagggccccggctacacgtacaag
gagctgctggt
gtggtactgcgacaacaccaacacccacggccccaagcgcatcatctgtgaggggcccaagaagaaagc
catgtggttcc
tgctcaccctgctcttcgccgccctcgtctgctggcagtggggcatcttcatcaggacctacttgagctgggagg
tcagc
gtctccctctccgtaggcttcaagaccatggacttccccgccgtcaccatctgcaatgctagccccttcaagtatt
ccaa
aatcaagcatttgctgaaggacctggatgagctgatggaagctgtcctggagagaatcctggctcctgagcta
agccatg
ccaatgccaccaggaacctgaacttctccatctggaaccacacacccctggtccttattgatgaacggaaccc
ccaccac
cccatggtccttgatctctttggagacaaccacaatggcttaacaagcagctcagcatcagaaaagatctgta
atgccca
cgggtgcaaaatggccatgagactatgtagcctcaacaggacccagtgtaccttccggaacttcaccagtgc
tacccagg
cattgacagagtggtacatcctgcaggccaccaacatctttgcacaggtgccacagcaggagctagtagag
atgagctac
cccggcgagcagatgatcctggcctgcctattcggagctgagccctgcaactaccggaacttcacgtccatct
tctaccc
tcactatggcaactgttacatcttcaactggggcatgacagagaaggcacttccttcggccaaccctggaact
gaattcg
gcctgaagttgatcctggacataggccaggaagactacgtccccttccttgcgtccacggccggggtcaggct
gatgctt
cacgagcagaggtcataccccttcatcagagatgagggcatctacG ccatgtcggggacagagacgtcca
tcggggtact
cgtggacaagcttcagcgcatgggggagccctacagcccgtgcaccgtgaatggttctgaggtccccgtcca
aaacttct
acagtgactacaacacgacctactccatccaggcctgtcttcgctcctgcttccaagaccacatgatccgtaac
tgcaac
tgtggccactacctgtacccactGccccgtggggagaaatactgcaacaaccgggacttcccagactgggc
ccattgcta
ctcagatctacagatgagcgtggcgcagagagagacctgcattggcatgtgcaaggagtcctgcaatgaca
cccagtaca
agatgaccatctccatggctgactggccttctgaggcctccgaggactggattttccacgtcttgtctcaggagc
gggac
caaagcaccaatatcaccctgagcaggaagggaattgtcaagctcaacatctActtccaagaatttaactatc
gcaccat
tgaagaatcagcagccaataacatcgtctggctgctctcgaatctgggtggccagtttggcttctggatggggg
gctctg
tgctgtgcctcatcgagtttggggagatcatcatcgactttgtgtggatcaccatcatcaagctggtggccttggc
caag
agcctacggcagcggcgagcccaagccagCtacgctggcccaccgcccaccgtggccgagctggtgga
ggcccacaccaactttggcttccagcctgacacggccccccgcagccccaacactgggccctaccccagtg
agcaggccctgcccatcccag
gcaccccgccccccaactatgactccctgcgtctgcagccgctggacgtcatcgagtctgacagtgagggtg
atgccatc
taa

SEQ ID NO: 3

Length 1950 nucleotides

DNA

Human hENaC gamma clone #3 coding sequence

atggcacccggagagaagatcaaagccaaaatcaagaagaatctgcccgtgacgggccctcaggcgcc
gaccattaaaga
gctgatgcggtggtactgcctcaacaccaacacccatggctgtcgccgcatcgtggtgtcccgcggccgtctg
cgccgcc
tcctctggatcgggttcacactgactgccgtggccctcatcctctggcagtgcgccctcctcgtcttctccttctata
ct
gtctcagtttccatcaaagtccacttccggaagctggattttcctgcagtcaccatctgcaacatcaacccctac
aagta
cagcaccgttcgccaccttctagctgacttggaacaggagaccagagaggccctgaagtccctgtatggcttt
ccagagt
cccggaagcgccgagaggcggagtcctggaactccgtctcagagggaaagcagcctagattctcccaccg
gattccgctg
ctgatctttgatcaggatgagaagggcaaggccagggacttcttcacagggAggaagcggaaagtcggcg
gtagcatcat
tcacaaggcttcaaatgtcatgcacatcgagtccaagcaagtggtgggattccaactgtgctcaaatgacacc
tccgact
gtgccacctacaccttcagctcgggaatcaatgccattcaggagtggtataagctacactacatgaacatcat
ggcacag
gtgcctctggagaagaaaatcaacatgagctattctgctgaggagctgctggtgacctgcttctttgatggagtg
tcctg
tgatgccaggaatttcacgcttttCcaccacccgatgcatgggaattgctatacAttcaacaacagagaaaatg
agacca
ttctcagcacctccatggggggcagcgaatatgggctgcaagtcattttgtacataaacgaagaggaataca
acccattc
ctcgtgtcctccactggagctaaggtgatcatccatcggcaggatgagtatcccttcgtcgaagatgtgggaac
agagat
tgagacagcaatggtcacctctataggaatgcacctgacagagtccttcaagctgagtgagccctacagtca
gtgcacgg
aggacgggagtgacgtgccaatcaggaacatctacaacgctgcctactcgctccagatctgccttcattcatg
cttccag
acaaagatggtggagaaatgtgggtgtgcccagtacagccagcctctacctcctgcagccaactactgcaa
ctaccagca
gcaccccaactggatgtattgttactaccaactgcatcgagcctttgtccaggaagagctgggctgccagtctg
tgtgca
aggaagcctgcagctttaaagagtggacactaaccacaagcctggcacaatggccatctgtggtttcggaga
agtggttg
ctgcctgttctcacttgggaccaaggccggcaagtaaacaaaaagctcaacaagacagacttgGccaaac
tcttgatatt
ctacaaagacctgaaccagagatccatcatggagagcccagccaacagtattgagatgcttctgtccaactt
cggtggcc
agctgggcctgtggatgagctgctctgttgtctgcgtcatcgagatcatcgaggtcttcttcattgacttcttctctat
c
attgcccgccgccagtggcagaaagccaaggagtggtgggcctggaaacaggctcccccatgtccagaa
gctccccgtag
cccacagggccaggacaatccagccctggatatagacgatgacctacccactttcaactctgctttgcacctg
cctccaG
ccctaggaacccaagtgcccggcacaccgccccccaaatacaataccttgcgcttggagagggccttttcca
accagctc
acagatacccagatgctAgatgagctctga

SEQ ID NO: 4

Length 669 amino acids

PRT

Human hENaC alpha clone #3-1-1 amino acid sequence

MEGNKLEEQDSSPPQSTPGLMKGNKREEQGLGPEPAAPQQPTAEEEALIE
FHRSYRELFEFFCNNTTIHGAIRLVCSQHNRMKTAFWAVLWLCTFGMMYW
QFGLLFGEYFSYPVSLNINLNSDKLVFPAVTICTLNPYRYPEIKEELEEL
DRITEQTLFDLYKYSSFTTLVAGSRSRRDLRGTLPHPLQRLRVPPPPHGA
RRARSVASSLRDNNPQVDWKDWKIGFQLCNQNKSDCFYQTYSSGVDAVRE
WYRFHYINILSRLPETLPSLEEDTLGNFIFACRFNQVSCNQANYSHFHHP
MYGNCYTFNDKNNSNLWMSSMPGINNGLSLMLRAEQNDFIPLLSTVTGAR
VMVHGQDEPAFMDDGGFNLRPGVETSISMRKETLDRLGGDYGDCTKNGSD
VPVENLYPSKYTQQVCIHSCFQESMIKECGCAYIFYPRPQNVEYCDYRKH
SSWGYCYYKLQVDFSSDHLGCFTKCRKPCSVTSYQLSAGYSRWPSVTSQE
WVFQMLSRQNNYTVNNKRNGVAKVNIFFKELNYKTNSESPSVTMVTLLSN
LGSQWSLWFGSSVLSWEMAELVFDLLVIMFLMLLRRFRSRYWSPGRGGRG
AQEVASTLASSPPSHFCPHPMSLSLSQPGPAPSPALTAPPPAYATLGPRP
SPGGSAGASSSTCPLGGP

SEQ ID NO: 5

Length 640 amino acids

PRT

Human hENaC beta clone #5 amino acid sequence

MHVKKYLLKGLHRLQKGPGYTYKELLVWYCDNTNTHGPKRIICEGPKKKA
MWFLLTLLFAALVCWQWGIFIRTYLSWEVSVSLSVGFKTMDFPAVTICNA
SPFKYSKIKHLLKDLDELMEAVLERILAPELSHANATRNLNFSIWNHTPL
VLIDERNPHHPMVLDLFGDNHNGLTSSSASEKICNAHGCKMAMRLCSLNR
TQCTFRNFTSATQALTEWYILQATNIFAQVPQQELVEMSYPGEQMILACL
FGAEPCNYRNFTSIFYPHYGNCYIFNWGMTEKALPSANPGTEFGLKLILD
IGQEDYVPFLASTAGVRLMLHEQRSYPFIRDEGIYAMSGTETSIGVLVDK
LQRMGEPYSPCTVNGSEVPVQNFYSDYNTTYSIQACLRSCFQDHMIRNCN
CGHYLYPLPRGEKYCNNRDFPDWAHCYSDLQMSVAQRETCIGMCKESCND
TQYKMTISMADWPSEASEDWIFHVLSQERDQSTNITLSRKGIVKLNIYFQ
EFNYRTIEESAANNIVWLLSNLGGQFGFWMGGSVLCLIEFGEIIIDFVWI
TIIKLVALAKSLRQRRAQASYAGPPPTVAELVEAHTNFGFQPDTAPRSPN
TGPYPSEQALPIPGTPPPNYDSLRLQPLDVIESDSEGDAI

SEQ ID NO: 6

Length 650 amino acids

PRT

Human hENaC gamma clone #3 amino acid sequence

MAPGEKIKAKIKKNLPVTGPQAPTIKELMRWYCLNTNTHGCRRIVVSRGR
LRRLLWIGFTLTAVALILWQCALLVFSFYTVSVSIKVHFRKLDFPAVTIC
NINPYKYSTVRHLLADLEQETREALKSLYGFPESRKRREAESWNSVSEGK
QPRFSHRIPLLIFDQDEKGKARDFFTGRKRKVGGSIIHKASNVMHIESKQ
VVGFQLCSNDTSDCATYTFSSGINAIQEWYKLHYMNIMAQVPLEKKINMS
YSAEELLVTCFFDGVSCDARNFTLFHHPMHGNCYTFNNRENETILSTSMG
GSEYGLQVILYINEEEYNPFLVSSTGAKVIIHRQDEYPFVEDVGTEIETA
MVTSIGMHLTESFKLSEPYSQCTEDGSDVPIRNIYNAAYSLQICLHSCFQ
TKMVEKCGCAQYSQPLPPAANYCNYQQHPNWMYCYYQLHRAFVQEELGCQ
SVCKEACSFKEWTLTTSLAQWPSWSEKWLLPVLTWDQGRQVNKKLNKTDL
AKLLIFYKDLNQRSIMESPANSIEMLLSNFGGQLGLWMSCSWCVIEIIEV
FFIDFFSIIARRQWQKAKEWWAWKQAPPCPEAPRSPQGQDNPALDIDDDL
PTFNSALHLPPALGTQVPGTPPPKYNTLRLERAFSNQLTDTQMLDEL

SEQ ID NO: 7

Length 1917 nucleotides

DNA

gi|1066456|gb|U38254.1|HSU38254 Human amiloride sensitive sodium

channel delta subunit (□NaCh) mRNA, complete coding sequence

ATGGCTGAGCACCGAAGCATGGACGGGAGAATGGAAGCAGCCACACGGG
GGGGCTCTCACCTCCAGGCTGCAGCCCAGACGCCCCCCAGGCCGGGGC
CACCATCAGCACCACCACCACCACCCAAGGAGGGGCACCAGGAGGGGCT
GGTGGAGCTGCCCGCCTCGTTCCGGGAGCTGCTCACCTTCTTCTGCACC
AATGCCACCATCCACGGCGCCATCCGCCTGGTCTGCTCCCGCGGGAACC
GGCTCAAGACGAOGTCCTGGGGGCTGCTGTCCCTGGGAGCCCTGGTCGC
GCTCTGCTGGCAGCTGGGGCTCCTCTTTGAGCGTGACTGGCACCGCCCG
GTCCTCATGGCCGTCTCTGTGCACTCGGAGCGCAAGCTGGTCCCGCTGG
TCACCCTGTGTGACGGGAACCCACGTCGGCCGAGTCCGGTCCTCCGCCA
TCTGGAGCTGCTGGACGAGTTTGCCAGGGAGAACATTGACTCCCTGTAGA
ACGTCAACCTCAGCAAAGGCAGAGCCGCCCTCTCCGCCACTGTCCCCCG
CCACGAGCCCCCGTTCCACCTGGACCGGGAGATCCGTCTGCAGAGGCTG
AGCCACTCGGGCAGCCGGGTCAGAGTGGGGTTCAGACTGTGCAACAGCA
CGGGCGGCGACTGCTTTTACCGAGGCTACACGTCAGGCGTGGCGGCTGT
CCAGGACTGGTACCACTTCCACTATGTGGATATCCTGGCCCTGCTGCGCG
CGGCATGGGAGGACAGCCACGGGAGCCAGGACGGCCACTTCGTCCTCTC
CTGCAGTTACGATGGCCTGGACTGCCAGGCCCGACAGTTCCGGACCTTC
CACCACCCCACCTACGGCAGCTGCTACACGGTCGATGGCGTCTGGACAG
CTCAGCGCCCCGGCATCACCCACGGAGTCGGCCTGGTCCTCAGGGTTGA
GCAGCAGCCTCACCTCCCTCTGCTGTCCACGCTGGCCGGCATCAGGGTC
ATGGTTCACGGCCGTAACCACACGCCCTTCCTGGGGCACCACAGCTTCA
GCGTCCGGCCAGGGACGGAGGCCACCATCAGCATCCGAGAGGACGAGG
TGCACCGGCTCGGGAGCCCCTACGGCCACTGCACCGCCGGCGGGGAAG
GCGTGGAGGTGGAGCTGCTACACAACACCTCCTACACCAGGCAGGCCTG
CCTGGTGTCCTGCTTCCAGCAGCTGATGGTGGAGACCTGCTCCTGTGGCT
ACTACCTCCACCCTCTGCCGGCGGGGGCTGAGTACTGCAGCTCTGCCCG
GCACCCTGCCTGGGGACACTGCTTCTACCGCCTCTACCAGGACCTGGAG
ACCCACCGGCTCCCCTGTACCTCCCGCTGCCCCAGGCCCTGCAGGGAGT
CTGCATTCAAGCTCTCCACTGGGACCTCCAGGTGGCCTTCCGCCAAGTCA
GCTGGATGGACTCTGGCCACGCTAGGTGAACAGGGGCTGCCGCATCAGA
GCCACAGACAGAGGAGCAGCCTGGCCAAAATCAAGATCGTCTACCAGGA
GCTCAACTACCGCTCAGTGGAGGAGGCGCCCGTGTACTCGGTGCCGCAG
CTGCTCTCCGCCATGGGCAGCCTCTACAGCCTGTGGTTTGGGGCCTCCG
TCCTCTCCCTCCTGGAGCTCCTGGAGCTGCTGCTCGATGCTTCTGCCCTC
ACCCTGGTGCTAGGCGGCCGCCGGCTCCGCAGGGCGTGGTTCTCCTGG
CCCAGAGCCAGCCCTGCCTCAGGGGCGTCCAGCTCAAGCCAGAGGCCA
GTCAGATGCCCCCGCCTGCAGGCGGCACGTCAGATGACCCGGAGCCCA
GCGGGCCTTCATCTCCCACCCGTCATGCTTCCAGCGGTTCTGGCCCGACT
CTCAGCCGAAGACAGCTGCGCTGGGCCCCAGCCCCTTGAGACTCTGGAC
ACCTGA

SEQ ID NO: 8

Length 638 nucleotides

PRT

gi|1710872|sp|P51172|SCAD_HUMAN Amiloride-sensitive sodium channel

delta-subunit amino acid sequence (Epithelial Na+ channel delta subunit)

(Delta ENaC) (Nonvoltage-gated sodium channel 1 delta subunit) (SCNED)

(Delta NaCh)

MAEHRSMDGRMEAATRGGSHLQAAAQTPPRPGPPSAPPPPPKEGHQEGLV
ELPASFRELLTFFCTNATIHGAIRLVCSRGNRLKTTSWGLLSLGALVALC
WQLGLLFERHWHRPVLMAVSVHSERKLLPLVTLCDGNPRRPSPVLRHLEL
LDEFARENIDSLYNVNLSKGRAALSATVPRHEPPFHLDREIRLQRLSHSG
SRVRVGFRLCNSTGGDCFYRGYTSGVAAVQDWYHFHYVDILALLPAAWED
SHGSQDGHFVLSCSYDGLDCQARQFRTFHHPTYGSCYTVDGVWTAQRPGI
THGVGLVLRVEQQPHLPLLSTLAGIRVMVHGRNHTPFLGHHSFSVRPGTE
ATISIREDEVHRLGSPYGHCTAGGEGVEVELLHNTSYTRQACLVSCFQQL
MVETCSCGYYLHPLPAGAEYCSSARHPAWGHCFYRLYQDLETHRLPCTSR
CPRPCRESAFKLSTGTSRWPSAKSAGWTLATLGEQGLPHQSHRQRSSLAK
INIVYQELNYRSVEEAPVYSVPQLLSAMGSLYSLWFGASVLSLLELLELL
LDASALTLVLGGRRLRRAWFSWPRASPASGASSIKPEASQMPPPAGGTSD
DPEPSGPHLPRVMLPGVLAGVSAEESWAGPQPLETLDT

What is claimed:

1. A mammalian cell-based high throughput assay for the profiling and screening of putative modulators of an epithelial sodium channel (ENaC) comprising:
contacting a test cell expressing alpha, beta and gamma subunits or delta, beta and gamma subunits or a variant, fragment or functional equivalent of each of these three subunits and preloaded with a membrane potential fluorescent dye or a sodium fluorescent dye with at least one putative modulator compound in the presence of sodium or lithium; and
monitoring anion mediated changes in fluorescence of the test cell in the presence of the putative modulator/ENaC interactions compared to changes in the absence of the modulator to determine the extent of ENaC modulation.

2. The assay method of claim 1 in which is anion is sodium.

3. The assay method of claim 1 in which the anion is lithium.

4. The assay method of claim 1 in which the test cell is selected from the group consisting of MDCK, HEK293, HEK293 T, BHK, COS, NIH3T3, Swiss3T3 and CHO.

5. The assay method of claim 4 in which the cell is an HEK293 cell.

6. The assay method of claim 4 wherein said HEK293 cell is an HEK293T cell.

7. The assay method of claim 1 in which a said method is used to identify a compound as one which particularly modulates taste based on a detectable change in fluorescence.

8. The assay method of claim 7 wherein said taste is salty taste.

9. The assay method of claim 1 in which said test cells are seeded onto a well of a multi-well test plate.

10. The assay method of claim 9 wherein said test cells are contacted with a putative modulator by adding said putative modulation to the well of said multi-well test plate.

11. The assay method of claim 10 wherein said test cells are loaded with a membrane potential dye that allows for changes in fluorescence to be detected.

12. The assay method of claim 11 wherein said test cell expresses each of the alpha, beta and gamma ENaC subunits.

13. The assay method of claim 12 wherein said subunits are respectively encoded by SEQ ID NO: 1, 2 and 3, or a fragment thereof, or a DNA sequence that hybridizes thereto and encodes a functional hENaC subunit.

14. The assay method of claim 1 wherein said subunits are encoded by SEQ ID NO: 1, 2 and 3.

15. The assay method of claim 1 wherein said test cell expresses hENaC beta, gamma and delta subunits or a fragment or variant thereof.

16. The assay method of claim 15 wherein said beta, gamma and delta subunits are respectively encoded by SEQ ID NO.: 2, 3 and 7.

17. The assay method of claim 1, wherein said ENaC subunits all comprise human ENaC subunits cloned from human kidney cDNA.

18. The assay method of claim 1, wherein said ENaC subunits comprise human ENaC subunits cloned from human lung cDNA.

19. The assay method of claim 1, wherein the ENaC is a human ENaC that is encoded by human ENaC DNA sequences cloned from human taste cell cDNA.

20. The assay of claim 1, wherein the ENaC is comprised of alpha (or delta), beta and gamma subunits and selected from the group consisting of: a naturally occurring human ENaC, an alternatively spliced human ENaC, a functional variant thereof, or combinations thereof.

21. The assay of claim 1 wherein a fluorescence plate reader is used to monitor changes in fluorescence.

22. The assay of claim 1 wherein a voltage imaging plate reader is used to monitor changes in fluorescence.

23. The assay of claim 1 wherein the membrane potential dye is selected from the group consisting of Molecular Devices Membrane Potential Kit (cat#R8034), Di-4-ANEPPS (Pyridinium, 4-(2-(6-(dibutylamino)-2-naphthalenyl)ethenyl)-1-(3-sulfopropyl))-, hydroxide, inner salt), DiSBACC4(2) (bis-(1,2-dibarbituric acid)-trimethine oxanol), DiSBAC4(3) (bis-(1,3-dibarbituric acid)-trimethine oxanol), CC-2-DMPE (Pacific Blue™ 1,2-dietradecanoyl-sn-glycerol-3-phosphoethanolmine, triethylammonium salt) and SBFI-AM (1,3-Benzenedicarboxylic acid, 4,4′-[1,4,10-trioxa-7,13-diazacyclopentadecane-7,13-diylbis(5-methoxy-6,12-benzofurandiyl)]bis-, tetrakis[(acetyloxy)methyl] ester; (Molecular probes).

24. A method for monitoring the activity of an epithelial sodium channel (ENaC) comprising:
providing a test cell transfected with a functional ENaC comprised of alpha (or delta), beta, and gamma ENaC subunits, splice variants, fragments and subunit combinations thereof;
seeding the test cell in the well of a multi-well plate and incubating for a time sufficient to reach at least about 70% confluence;
dye-loading the seeded test cell with a membrane potential dye in the well of the multi-well plate;
contacting the dye-loaded test cell with at least one putative modulating compound and sodium in the well of the multi-well plate; and
monitoring any changes in fluorescence of the membrane potential dye due to modulator/ENaC interactions using a fluorescence plate reader or voltage intensity plate reader.

25. The method of claim 24 wherein said tests cell is an HEK293 cell.

26. The method of claim 24 wherein said test cell is a HEK293T cell.

27. The method of claim 24 wherein said alpha, beta and gamma subunits are encoded by SEQ ID NO.: 1, 2 and 3 respectively.

28. The method of claim 24 wherein said delta, beta and gamma subunits are encoded by SEQ ID NO.: 7, 2 and 3 respectively.

29. The method of claim 28 wherein the test cell is HEK293.

30. The method of claim 24, wherein the test cell is dye-loaded by adding the membrane potential dye to the well of the multi-well plate with the test cell seeded therein and incubating for a period of time sufficient to allow for equilibration of the dye through the membrane of the test cell.

31. The method of claim 30, wherein the membrane potential dye is added to the well of the multi-well plate at a concentration of about 2 μM to about 5 μM of the final concentration.

32. The method of claim 24, wherein the membrane potential dye is selected from the group consisting of Molecular Devices Membrane Potential Kit (cat# R8034), Di-4-ANEPPS (Pyridinium, 4-(2-(6-(dibutylamino)-2-naphthalenyl)ethenyl)-1-(3-sulfopropyl)-, hydroxide, inner salt), DiSBAC4(2) (bis-(1,2-dibarbituric acid)-trimethine oxanol), DiSBAC4(3) (bis-(1,3-dibarbituric acid)-trimethine oxanol), CC-2-DMPE (Pacific Blue™ 1,2-ditetradecanoyl-sn-glycero-3-phosphoethanolamine, triethylammonium salt). and SBFI-AM (1,3-Benzenedicarboxylic acid, 4,4′-[1,4,10-trioxa-7,13-diazacyclopentadecane-7,13-diylbis(5-methoxy-6,12-benzofurandiyl)]bis-, tetrakis[(acetyloxy)methyl] ester; (Molecular probes).

33. The method of claim 24, wherein the ENaC is a human ENaC encoded by ENaC subunit DNAs cloned from human kidney cDNA.

34. The method of claim 24, wherein the ENaC is a human ENaC encoded by ENaC subunits DNAs cloned from human lung cDNA.

35. The method of claim 24, wherein the ENaC is a human ENaC encoded by ENaC subunits DNAs cloned from human taste cell cDNA.

36. The method of claim 24, wherein the ENaC is selected from the group consisting of: a naturally occurring human ENaC subunit, an alternatively spliced human ENaC subunit, a functional variant thereof and combinations where the cells expresses alpha, beta and gamma subunits.

37. The method of claim 24, wherein the ENaC comprises alpha (or delta), beta and gamma subunits of a naturally occurring human ENaC, or an alternatively spliced version thereof or combinations thereof.

38. The method of claim 24, wherein the test cell is selected from the group consisting of MDCK, HEK293, HEK293T, COS, BHK, NIH3T3, Swiss3T3and CHO cell.

39. The method of claim 24 wherein the test cells are grown to 80% confluence.

40. A method for identifying a salty taste modulating compound comprising: providing a test cell transfected with a functional human ENaC; splice variant, chimera or fragment thereof;
seeding the test cell in the well of a multi-well plate and incubating for a time sufficient to reach at least about 70% confluence;
dye-loading the seeded test cell with a membrane potential dye in the well of the multi-well plate;
contacting the dye-loaded test cell with at least one putative modulating compound and sodium in the well of the multi-well plate;
monitoring any changes in fluorescence of the membrane potential dye due to modulator/ENaC interactions using a fluorescence plate reader or voltage intensity plate reader; and
identifying the at least one putative modulator as a salty taste modulating compound based on the monitored changes in fluorescence.

41. The method of claim 40 further comprising evaluating the identified ENaC modulating compound for effects on salty taste perception.

42. The method of claim 40 wherein said test cell is selected from the group consisting of MDCK, HEK293, HEK2933T, COS, BHK, NIH3T3, Swiss3T3 and CHO.

43. The method of claim 42 wherein said test cell is an HEK293 cell.

44. The method of claim 43 wherein said test cell is a HEK2933T cell.

45. The method of claim 41 in which the cell is an HEK293 cell.

46. The method of claim 45 wherein said HEK293 cell is an HEK293T cell.

47. The method of claim 40 in which a said method is used to identify a compound as one which particularly modulates taste based on a detectable change in fluorescence.

48. The method of claim 47 wherein said taste is salty taste.

49. The assay method of claim 40 in which said test cells are seeded on to a well of a multi-well test plate and grown to about 80% confluence.

50. The method of claim 49 wherein said test cells are contacted with a putative modulator by adding said putative modulation to the well of said multi-well test plate.

51. The method of claim 50 wherein said test cells are loaded with a membrane potential dye that allows for changes in fluorescence to be detected.

52. The method of claim 51 wherein said test cell expresses each of the alpha, beta and gamma ENaC subunits.

53. The method of claim 52 wherein said subunits are respectively encoded by SEQ ID NO: 1, 2 and 3, or a fragment thereof, or a DNA sequence that hybridizes thereto and encodes a functional hENaC subunit.

54. The method of claim 53 wherein said subunits are encoded by SEQ ID NO: 1, 2 and 3.

55. The method of claim 40 wherein said test cell expresses hENaC beta, gamma and delta subunits or a fragment or variant thereof.

56. The method of claim 15 wherein said beta, gamma and delta subunits are respectively encoded by SEQ ID NO.: 2, 3 and 7.

57. The assay of claim 40, wherein said ENaC subunits all comprise human ENaC subunits cloned from human kidney cDNA.

58. The assay of claim 40, wherein said ENaC subunits all comprise human ENaC subunits cloned from human lung cDNA.

59. The assay of claim 40, wherein the ENaC is a human ENaC that is encoded by human ENaC DNA sequences cloned from human taste cell cDNA.

60. The assay of claim 40, wherein the ENaC is comprised of alpha (or delta), beta and gamma subunits and selected from the group consisting of: a naturally occurring human ENaC, an alternatively spliced human ENaC, a functional variant thereof, or subunit combinations thereof.

61. The assay of claim 40 wherein a fluorescence plate reader is used to monitor changes in fluorescence.

62. The assay of claim 40 wherein a voltage imaging plate reader is used to monitor changes in fluorescence.

63. The assay of claim 40 wherein the membrane potential dye is selected from the group consisting of Molecular Devices Membrane Potential Kit (cat#R8034), Di-4-ANEPPS (Pyridinium, 4-(2-(6-(dibutylamino)-2-naphthalenyl)ethenyl)-1-(3-sulfopropyl))-, hydroxide, inner salt), DiSBACC4(2) (bis-(1,2-dibarbituric acid)-trimethine oxanol), DiSBAC4(3) (bis-(1,3-dibarbituric acid)-trimethine oxanol), CC-2-DMPE (Pacific Blue™ 1,2-dietradecanoyl-sn-glycerol-3-phosphoethanolmine, triethylammonium salt) and SBFI-AM (1,3-Benzenedicarboxylic acid, 4,4′-[1,4,10-trioxa-7,13-diazacyclopentadecane-7,13-diylbis(5-methoxy-6,12-benzofurandiyl)]bis-, tetrakis[(acetyloxy)methyl] ester; (Molecular probes).

64. A recombinant mammalian cell that stably or transiently expresses a functional human ENaC.

65. The mammalian cell of claim 64 which is selected from the group consisting of MDCK, HEK293, HEK293T, COS, BHK, NIH3T3, Swiss3T3 and CHO.

66. The mammalian cell of claim 65 which are HEK293 cell.

67. The mammalian cell of claim 64 which expresses an alpha (or delta), beta and gamma hENaC subunit, or a variant, fragment or chimera or combinations thereof.

68. The mammalian cell of claim 67 which is a HEK293T cell that expresses the nucleic acid sequences contained in SEQ ID NO: 1 (or DELTA SEQ), 2 and 3 or nucleic acid sequences that hybridize under high stringency hybridization conditions to each of said nucleic acid sequences.

69. The mammalian cell of claim 64 which expresses the alpha, beta and gamma subunits encoded by SEQ ID NO: 1, 2 and 3.

70. The mammalian cell of claim 64 which expresses the beta, gamma and delta ENaC subunits encoded by SEQ ID NO: 2, 3 and 7 respectively.

71. The mammalian cell of claim 64 which transiently expresses said ENaC.

72. The mammalian cell of claim 64 which transiently expresses said ENaC.

73. The mammalian cell of claim 64 which inducibly expresses said ENaC.

74. A composition which comprises a mammalian cell according to any one of claims 64-73 and at least one membrane potential dye.

75. The composition of claim 74 wherein said membrane potential dye is selected from the group consisting of Molecular Devices Membrane Potential Kit (cat# R8034), Di-4-ANEPPS (Pyridinium, 4-(2-(6-(dibutylamino)-2-naphthalenyl)ethenyl)-1-(3-sulfopropyl)-, hydroxide, inner salt), DiSBAC4(2) (bis-(1,2-dibarbituric acid)-trimethine oxanol), DiSBAC4(3) (bis-(1,3-dibarbituric acid)-trimethine oxanol), CC-2-DMPE (Pacific Blue™ 1,2-ditetradecanoyl-sn-glycero-3-phosphoethanolamine, triethylammonium salt). and SBFI-AM (1,3-Benzenedicarboxylic acid, 4,4′-[1,4,10-trioxa-7,13-diazacyclopentadecane-7,13-diylbis(5-methoxy-6,12-benzofurandiyl) ]bis-, tetrakis[(acetyloxy)methyl] ester; (Molecular probes).

76. A method for identifying a compound that modulates hENaC comprising;
(i) contacting a mammalian cell according to claim 64 with a candidate compound that positively modulates an epithelial sodium channel; and
(ii) determining whether said candidate compound modulates or binds to said hENaC and/or affects the activity of said hENaC.

77. The method of claim 76 wherein said mammalian cell is selected from the group consisting of MDCK, BHK, HEK293, HEK293T, COS, NIH3T3, Swiss3T3 and CHO.

78. The method of claim 76 wherein said mammalian cell is an HEK293 cell.

79. The method of claim 78 wherein said cell transiently or stably expresses the alpha (or delta), beta and gamma ENaC subunits.

80. The method of claim 76 wherein said mammalian cell is comprised in a multi-well test plate device.

81. The method of claim 80 wherein said mammalian cell is loaded with a membrane potential dye, contacted with a putative ENaC modulator and sodium, and change in fluorescence monitored using a voltage intensity plate reader or fluorescence plate reader.

82. The method of claim 81 wherein said mammalian cells are grown to about 80% confluence.

83. The method of claim 82 wherein the membrane potential dyes are CC2-DMPVE or DiSBAC2(3) and ESS-CY4.

84. The method of claim 83 wherein the dye is comprised in a loading buffer.

85. The method of claim 84 wherein after cells are loaded with the dye variation of cell density is evaluated.

86. The method of claim 83 which includes the use of a positive or negative control compound that modulates ENaC.

87. The method of claim 86 wherein said control is a compound known to inhibit ENaC.

88. The method of claim 86 wherein said compound is amiloride or Phenamil.
